SEQUENCE LISTING (32 sequences)

SEQ ID NO: 1 - DNA, 4272 bp, Artificial Sequence
  nucleotide sequence encoding AtPDR12 protein
atggagggaactagttttcaccaagcgagtaatagtatgagaagaaactcatcggtgtggaagaaagattcaggaagggagattttctcgaggtcatctagagaagaagacgatgaagaagctttgagatgggctgctcttgagaagcttcccacttttgatcgtctcaggaaaggaatcctaactgcctcacatgccggaggacccatcaacgagatcgatattcagaagcttgggtttcaagatactaagaaactgctagagaggctcatcaaagtcggtgacgatgagcatgagaaactcctctggaaactcaagaaacgtatcgatagagttggaatcgatcttccgacaatagaagttcggtttgatcatctaaaagttgaagcagaggttcatgttggaggcagagctttacctacgttcgtcaatttcatctccaattttgctgataagttcctgaatactctgcatcttgttccgaaccgaaagaagaagttcactatactcaacgacgtcagcggaatcgtcaagcctggcaggatggctctgcttttgggtcctccaagttctgggaaaacgaccctcttgcttgccttggcgggaaagcttgatcaagaactaaagcaaactggaagagtgacatacaatggtcatggaatgaacgagtttgtgccacaaagaacagctgcatatatcggccaaaacgatgttcatatcggtgagatgactgttcgtgagacttttgcttacgcagctcgcttccaaggtgttggttcgcgttatgacatgttgacagagttggcaagaagagagaaagaagcaaacatcaaacctgaccctgatattgatatattcatgaaggcgatgtcaacagcaggtgaaaaaacaaatgtgatgacagattatatcctcaagatcttaggacttgaggtctgtgcagacactatggtcggcgatgatatgttgagaggcatctccggaggacaaaagaagcgtgtcactactggtgaaatgctggttggaccgtctagggctctgttcatggatgagatatcgactggtttagatagttcaacgacttaccagatagtgaactccctcagaaactatgttcatatcttcaatgggacagctctgatctctctccttcagcctgcgccagagacattcaatctcttcgatgatatcattctcattgcagaaggcgagatcatctacgagggccctcgtgatcacgttgtggagttctttgagaccatgggattcaaatgtcctccaagaaaaggcgttgctgatttccttcaagaagtgacatcaaagaaagaccaaatgcagtactgggcacgacgtgatgagccttacaggttcattagagtgagagagtttgcagaggcgtttcaatcattccacgttggccggagaatcggagatgagcttgctttgccctttgacaagacaaagagccatccggctgctctaaccaccaagaaatacggagttgggattaaagaacttgtcaagaccagcttctcaagagaatacttactcatgaaaagaaactcctttgtttactacttcaagtttggacaactgctggtaatggcatttttgacaatgacgttgttctttcggacggagatgcaaaagaagactgaggttgatgggagtctctacactggagccttgttcttcatccttatgatgctcatgttcaatggaatgtctgaactttcaatgaccatagcaaaacttcctgtgttttacaaacaaagagatctcctcttctaccctgcatgggtgtactctctgcctccttggctcctcaagatacctataagcttcatggaagccgctctcacaacattcatcacttactatgtcatcggctttgatcccaacgttggaaggctgtttaagcagtatattctcctcgtgctcatgaaccaaatggcttcagcattgtttaagatggtggcagcattgggaagaaacatgatcgttgcaaatacatttggtgcatttgcgatgctcgtcttctttgccttgggtggtgtggtactttcacgagacgacattaagaagtggtggatatggggttactggatctccccaataatgtatggacagaacgcgatcctagccaatgagttctttggacacagctggagtcgagctgtcgaaaactcgagcgaaacacttggagttactttccttaagtctcgtgggttcttaccccatgcatactggtactggattggaactggagccttacttgggttcgtcgtgttattcaattttggtttcacgctggctctgacgtttctgaactccttgggaaagcctcaagctgttattgcagaagagcctgcgagtgatgagacagaacttcagtctgctaggtcagaaggtgtagttgaagctggtgccaataagaaaagagggatggtgcttccatttgagccacattcaattaccttcgacaatgttgtatactcagttgacatgccccaggaaatgatagagcaaggcacacaagaagacagacttgtcctgttgaaaggtgtgaatggtgcattcaggccaggcgtgctcacggctctcatgggtgtctctggagctggcaaaaccactctgatggatgttcttgccggaaggaaaaccggtggttatattgatggcaacatcaccatttccggttaccctaagaatcaacaaacatttgcccgtatctcaggatactgtgaacaaactgatatccattccccacatgtcactgtttacgagtccttggtttactcagcctggctccgattacctaaagaagttgataaaaacaagagaaagatattcatagaggaagtgatggagctggtggagttaacgccgctgaggcaagcactggttggactacctggtgagagcggtttgtcaacagagcaaagaaagagactgaccattgcggtggagctggttgcaaatccttccatcatattcatggatgaacctacttcaggattggatgcacgagctgctgccatcgttatgaggactgtaaggaacacagttgacactggtagaacagtcgtctgcaccattcaccagcctagcatcgacatctttgaagcctttgatgagttgttcctacttaagcgtggaggtgaggagatatacgttggacctcttggccacgaatcaacccatttgatcaactattttgagagtattcaaggaatcaacaagatcacagaaggatacaacccagcaacctggatgcttgaagtctcaaccacatctcaagaagcggctttaggagtcgatttcgcccaagtctacaaaaattcagaactttacaagagaaacaaggagctaatcaaggagctaagccagccagctccaggatcaaaagatttatatttcccaacacaatactctcaatcgttcttgacacaatgtatggcttctctatggaaacaacactggtcctactggagaaatcctccttacacagccgtgagattcctcttcacaatcggcattgctcttatgttcggcacaatgttctgggaccttggaggcaaaacgaaaacgagacaggatttatcgaatgcaatgggttcaatgtacacagctgttctcttcctcggattacaaaacgcagcttcagtgcaaccagtcgtcaacgtcgaaagaactgtcttttaccgagaacaagccgccggaatgtactccgccatgccttatgctttcgctcaggttttcatcgagatcccatacgttctcgtgcaagcgatagtgtacggtctcatagtgtacgctatgataggattcgagtggacggcggtgaagttcttctggtacctcttctttatgtacggatcattcttaactttcaccttctacggaatgatggctgtagctatgacgcctaaccaccacatcgcctccgtcgtctcctccgctttctacggcatctggaatctcttctccggcttcctcatccctcgtcccagtatgcctgtgtggtgggaatggtactactggctttgcccagttgcatggacattgtatggattaatcgcatcacagttcggtgatattacagaacctatggcagatagtaatatgagtgtgaagcaattcattagagaattctatggatatagagaaggtttcttgggtgtggttgccgccatgaacgtcatctttcctttgctctttgccgttatctttgctatcggaatcaagagtttcaatttccaaaaacgatag

SEQ ID NO: 2 - protein, 1423 aa, Artificial Sequence
  amino acid sequence of AtPDR12 protein
MEGTSFHQASNSMRRNSSVWKKDSGREIFSRSSREEDDEEALRWAALEKLPTFDRLRKGILTASHAGGPINEIDIQKLGFQDTKKLLERLIKVGDDEHEKLLWKLKKRIDRVGIDLPTIEVRFDHLKVEAEVHVGGRALPTFVNFISNFADKFLNTLHLVPNRKKKFTILNDVSGIVKPGRMALLLGPPSSGKTTLLLALAGKLDQELKQTGRVTYNGHGMNEFVPQRTAAYIGQNDVHIGEMTVRETFAYAARFQGVGSRYDMLTELARREKEANIKPDPDIDIFMKAMSTAGEKTNVMTDYILKILGLEVCADTMVGDDMLRGISGGQKKRVTTGEMLVGPSRALFMDEISTGLDSSTTYQIVNSLRNYVHIFNGTALISLLQPAPETFNLFDDIILIAEGEIIYEGPRDHVVEFFETMGFKCPPRKGVADFLQEVTSKKDQMQYWARRDEPYRFIRVREFAEAFQSFHVGRRIGDELALPFDKTKSHPAALTTKKYGVGIKELVKTSFSREYLLMKRNSFVYYFKFGQLLVMAFLTMTLFFRTEMQKKTEVDGSLYTGALFFILMMLMFNGMSELSMTIAKLPVFYKQRDLLFYPAWVYSLPPWLLKIPISFMEAALTTFITYYVIGFDPNVGRLFKQYILLVLMNQMASALFKMVAALGRNMIVANTFGAFAMLVFFALGGVVLSRDDIKKWWIWGYWISPIMYGQNAILANEFFGHSWSRAVENSSETLGVTFLKSRGFLPHAYWYWIGTGALLGFVVLFNFGFTLALTFLNSLGKPQAVIAEEPASDETELQSARSEGVVEAGANKKRGMVLPFEPHSITFDNVVYSVDMPQEMIEQGTQEDRLVLLKGVNGAFRPGVLTALMGVSGAGKTTLMDVLAGRKTGGYIDGNITISGYPKNQQTFARISGYCEQTDIHSPHVTVYESLVYSAWLRLPKEVDKNKRKIFIEEVMELVELTPLRQALVGLPGESGLSTEQRKRLTIAVELVANPSIIFMDEPTSGLDARAAAIVMRTVRNTVDTGRTVVCTIHQPSIDIFEAFDELFLLKRGGEEIYVGPLGHESTHLINYFESIQGINKITEGYNPATWMLEVSTTSQEAALGVDFAQVYKNSELYKRNKELIKELSQPAPGSKDLYFPTQYSQSFLTQCMASLWKQHWSYWRNPPYTAVRFLFTIGIALMFGTMFWDLGGKTKTRQDLSNAMGSMYTAVLFLGLQNAASVQPVVNVERTVFYREQAAGMYSAMPYAFAQVFIEIPYVLVQAIVYGLIVYAMIGFEWTAVKFFWYLFFMYGSFLTFTFYGMMAVAMTPNHHIASVVSSAFYGIWNLFSGFLIPRPSMPVWWEWYYWLCPVAWTLYGLIASQFGDITEPMADSNMSVKQFIREFYGYREGFLGVVAAMNVIFPLLFAVIFAIGIKSFNFQKR

SEQ ID NO: 3 - DNA, 4281 bp, Artificial Sequence
  nucleotide sequence encoding AtPDR3 protein
atggcggcggcttcgaatgggagtgagtatttcgaattcgatgtagaaacaggaagagaatcgttcgcgcggccgtctaacgccgagacagtggagcaagatgaggaagatctacggtgggcggcgatcggaagattaccgtcgcagagacaagggactcataatgcgattctgcgtcggtcgcaaacgcaaacgcagacttctggttacgctgatggaaacgtggttcaaacgattgatgtgaagaaattggatcgtgcggatcgtgagatgttggttcgtcaagctcttgctactagtgatcaagataatttcaagcttctctctgctattaaagaacgtcttgatcgagttggtatggaggttccgaaaatcgaagtgcggttcgagaatttgaatattgaagctgatgttcaagctggtacaagagctttgcctactttggttaatgtctctcgtgatttctttgagcgttgcttaagcagcttgaggataatcaagcctaggaaacacaagcttaatattttgaaagatattagtgggattatcaaaccaggaaggatgactttgttgttaggaccaccgggttccggaaaatcgacgttgcttcttgctcttgcagggaagcttgataagagtttaaagaaaacgggtaacattacctataatggagagaatcttaacaagtttcatgttaaaagaacttcagcatatataagtcaaacagataatcacattgctgaactcactgttcgtgaaactcttgatttcgctgcgagatgtcaaggcgcaagcgaaggatttgcaggttacatgaaagatctaacccgattagagaaagagaggggtatacgtccttcttctgagattgatgcgtttatgaaggctgcttctgtcaaaggcgaaaagcatagtgtttcgacagattatgtgcttaaagtacttggtcttgatgtatgttctgatacaatggttggtaatgatatgatgagaggtgtttcaggaggtcaaaggaaaagagttacaacaggagaaatgactgttgggccaagaaaaacgttgtttatggatgaaatatctactggtcttgatagttcaacaactttccagattgtcaaatgtatcagaaacttcgttcatctaatggatgcaactgttcttatggcacttcttcagcctgcaccagaaacatttgacttgtttgatgacttaattcttctatcagaaggttacatggtatatcaaggtcctcgagaagatgtgatagccttttttgagtctctaggattccgtctcccaccgcgtaaaggtgtggcagattttctccaagaggtgacctctaaaaaggatcaagctcaatactgggcagatccttcgaagccatatcagttcattcctgtctcagacatagcggctgcattccgtaactcgaaatacgggcatgctgcggattccaagctggctgcaccatttgacaagaaatctgcggatccttcagctttatgcagaacaaaatttgccatttcaggatgggaaaaccttaaagtttgctttgtacgagaattattattgatcaaacggcacaaatttctttacacttttaggacatgccaggttggatttgtgggacttgtcacagcgacagtgttcttgaaaactagattacacccaacaagcgaacaatttggcaatgagtatctatcttgccttttcttcgggctagtccacatgatgttcaacggtttctctgaactacctctcatgatatcgcgtctcccggttttttacaagcaaagggataactcctttcatcctgcttggtcatggtctattgccagctggctcttgcgtgtgccttactctgtccttgaagctgttgtctggagtggtgtagtatacttcactgtgggacttgctccctcagcaggcagatttttccgatacatgttacttctcttctcggtgcatcaaatggctctcggtttatttcgtatgatggcttctctagcaagggacatggtcattgccaatacattcggatctgcagcaatcttaatagtgttcttgcttggtggattcgttattccaaaagctgatattaaaccttggtgggtttggggtttttgggtgtcgccgttatcctatggacaacgtgccattgcggtcaatgaattcacagccacacggtggatgacgccatcagctatatctgatactacaattggactcaaccttctaaagctacgtagctttccaacgaatgactattggtattggattggaatcgcggttctcattggttatgcaattctattcaataacgtagtaactctcgccttggcttatcttaaccctctaagaaaagctcgagcagttgttttagacgatcccaatgaagaaactgctttagtagcagatgcaaatcaagtaataagtgagaaaaagggaatgatccttccattcaaaccattaacaatgactttccacaatgtcaactattatgttgacatgccaaaggaaatgcgttctcaaggtgtaccagagacaagactacaactgttatcaaacgtgagtggagtgttctcacctggagttcttacagctttagttggatcaagcggtgcgggaaaaactacgttgatggatgttcttgcgggtcgaaagacaggtggatataccgagggagatattagaatctctggtcacccaaaagaacaacaaacgtttgctaggatctctggatatgttgagcagaacgatatacactctcctcaagtcacagttgaagagtccttatggttttcggctagccttcgtcttcctaaggagatcaccaaagaacagaaaaaggaatttgtggaacaagtcatgagacttgtagagcttgacactctgagatatgctttagtaggtttacctggtaccacgggtctttctactgagcagaggaaacgtctaacaattgcggttgagttagtagcaaatccatcaattattttcatggatgaaccaacatctggacttgatgcaagagcagcagcaattgtgatgagaactgttagaaacactgttgatactggaagaacagtggtttgcaccattcatcaacctagcattgacatttttgaggcctttgacgagctgcttctaatgaaacgaggaggacaggttatatatggtgggaaattgggtacacactcgcaggttctggtagattactttcaggggattaacggagtacctccaatctcaagtggttacaatccagcaacttggatgcttgaagtaactacacctgctttggaagagaaatataacatggagtttgcagatttatacaagaaatctgaccagtttagagaagtagaggcaaacatcaagcaactcagtgttccaccagaaggctcagaaccaataagctttacatcaagatactcacaaaaccaactatctcaatttctactctgtctctggaaacagaaccttgtctactggagaagtccagaatacaatctcgtgagattagtcttcacaacgatcgctgcgtttatactgggaacagttttctgggacattggttccaagaggacttcctcacaggatttgatcaccgtaatgggagctctctactcggcttgtctgtttcttggagttagtaacgcttcatcagtacaaccaatcgtttcaatcgaaagaacagttttctacagagagaaagccgctggtatgtatgctccaattccatatgcagcagctcagggtcttgtggagataccttacattctgacccaaaccattctctacggtgtcatcacatacttcaccattggctttgaaagaacgttcagtaagtttgtactgtatttggtgttcatgttcctcacattcacctacttcaccttctatggcatgatggcggttggtctcaccccgaatcagcatttagccgctgtgatctcctctgcgttttactctctctggaatctcctatccggtttcctcgtccaaaagcctttgattccagtgtggtggatatggttctattacatatgtccagtggcgtggacactacaaggagtaatcctttcgcagcttggtgacgtggaaagcatgatcaacgagccattgtttcatggcacggtgaaggagtttattgaatattacttcggctacaagccaaacatgattggtgtatctgctgctgttcttgtcggtttttgtgctctcttcttctcagcatttgcactttcagtcaaatacctcaacttccagagaagataa

SEQ ID NO: 4 - protein, 1426 aa, Artificial Sequence
  amino acid sequence of AtPDR3 protein
MAAASNGSEYFEFDVETGRESFARPSNAETVEQDEEDLRWAAIGRLPSQRQGTHNAILRRSQTQTQTSGYADGNVVQTIDVKKLDRADREMLVRQALATSDQDNFKLLSAIKERLDRVGMEVPKIEVRFENLNIEADVQAGTRALPTLVNVSRDFFERCLSSLRIIKPRKHKLNILKDISGIIKPGRMTLLLGPPGSGKSTLLLALAGKLDKSLKKTGNITYNGENLNKFHVKRTSAYISQTDNHIAELTVRETLDFAARCQGASEGFAGYMKDLTRLEKERGIRPSSEIDAFMKAASVKGEKHSVSTDYVLKVLGLDVCSDTMVGNDMMRGVSGGQRKRVTTGEMTVGPRKTLFMDEISTGLDSSTTFQIVKCIRNFVHLMDATVLMALLQPAPETFDLFDDLILLSEGYMVYQGPREDVIAFFESLGFRLPPRKGVADFLQEVTSKKDQAQYWADPSKPYQFIPVSDIAAAFRNSKYGHAADSKLAAPFDKKSADPSALCRTKFAISGWENLKVCFVRELLLIKRHKFLYTFRTCQVGFVGLVTATVFLKTRLHPTSEQFGNEYLSCLFFGLVHMMFNGFSELPLMISRLPVFYKQRDNSFHPAWSWSIASWLLRVPYSVLEAVVWSGVVYFTVGLAPSAGRFFRYMLLLFSVHQMALGLFRMMASLARDMVIANTFGSAAILIVFLLGGFVIPKADIKPWWVWGFWVSPLSYGQRAIAVNEFTATRWMTPSAISDTTIGLNLLKLRSFPTNDYWYWIGIAVLIGYAILFNNVVTLALAYLNPLRKARAVVLDDPNEETALVADANQVISEKKGMILPFKPLTMTFHNVNYYVDMPKEMRSQGVPETRLQLLSNVSGVFSPGVLTALVGSSGAGKTTLMDVLAGRKTGGYTEGDIRISGHPKEQQTFARISGYVEQNDIHSPQVTVEESLWFSASLRLPKEITKEQKKEFVEQVMRLVELDTLRYALVGLPGTTGLSTEQRKRLTIAVELVANPSIIFMDEPTSGLDARAAAIVMRTVRNTVDTGRTVVCTIHQPSIDIFEAFDELLLMKRGGQVIYGGKLGTHSQVLVDYFQGINGVPPISSGYNPATWMLEVTTPALEEKYNMEFADLYKKSDQFREVEANIKQLSVPPEGSEPISFTSRYSQNQLSQFLLCLWKQNLVYWRSPEYNLVRLVFTTIAAFILGTVFWDIGSKRTSSQDLITVMGALYSACLFLGVSNASSVQPIVSIERTVFYREKAAGMYAPIPYAAAQGLVEIPYILTQTILYGVITYFTIGFERTFSKFVLYLVFMFLTFTYFTFYGMMAVGLTPNQHLAAVISSAFYSLWNLLSGFLVQKPLIPVWWIWFYYICPVAWTLQGVILSQLGDVESMINEPLFHGTVKEFIEYYFGYKPNMIGVSAAVLVGFCALFFSAFALSVKYLNFQRR

SEQ ID NO: 5 - DNA, 4263 bp, Artificial Sequence
  nucleotide sequence encoding AtPDR4 protein
atgtggaactcggcggagaacgcattctcgcgttcgacgtcgtttaaagacgaaatcgaagacgaagaagagcttcgttgggcagctttacaacgtcttccaacttattcacgaatccgccgcgggattttcagagatatggttggtgagcctaaagagatccaaattggtaacctagaagctagcgaacaacgtcttcttctcgaccgtcttgtgaattccgttgaaaatgatcctgaacagttcttcgctcgtgttaggaagagattcgacgctgtggatttgaaatttccgaagattgaagttcgatttcagaatctgatggtagaatcatttgttcatgttggaagcagagcattaccaacaattcccaatttcatcattaacatggcagaaggtttgttgaggaacattcatgtgattggtggaaaaaggaacaaattaacgattttagatggtatcagtggtgttattagaccttcaagattgacactactactaggtcctccaagttcagggaagacaacattactcttagctttagctggacgtcttggaactaatcttcagacatcagggaaaataacgtacaatggctacgatctcaaagagataatcgcgccaaggacgtccgcatatgtgagccaacaggattggcatgtggcggagatgacagtgaggcagactcttgagtttgctggtcgttgtcaaggtgttggattcaaatatgatatgcttttggagttagcaagaagggaaaagcttgcaggaattgtacctgatgaagatcttgatatatttatgaagtctttagctctcgggggaatggagacaagcctcgtcgtggagtatgttatgaagattctaggattggacacatgtgcggacacattggttggagatgagatgattaaaggaatttcagggggacagaagaaacggctaacaacaggggaactattagttggtccagcaagagtccttttcatggatgaaatatcaaatggtttagatagttcgacgactcatcagattataatgtatatgaggcattctactcatgcgctcgaaggaaccaccgtcatttctttactccaaccgtctcccgagacttacgagttgtttgatgatgttatccttatgtctgagggacagattatataccagggacctcgtgacgaggttcttgatttcttttcttcgttgggatttacttgccctgatagaaaaaatgttgcagatttcttgcaagaggtcacctcaaagaaagatcagcagcagtactggtcagtccctttccggccttatcgctatgtacctcctggaaaatttgctgaagctttccgttcatatccaactggtaagaagttggcaaaaaaacttgaggttccatttgataaacggttcaatcattcagccgcgttatcgacatcccaatatggtgttaagaagagtgaacttctcaagatcaactttgcctggcagaagcaactgatgaagcaaaatgcatttatttatgtgttcaagtttgttcagcttcttttagttgctttgatcacgatgactgtcttttgccgaacaacaatgcatcacaacactattgatgatggcaacatatatcttgggtcactttacttttccatggtcatcattctctttaacggattcaccgaggttccaatgctagtagccaaacttcccgttctttataagcacagagacttgcatttttacccaagctgggcttatacattaccttcctggcttttgagtatccctacttcaataatagaatctgccacatgggtggcagtaacatattatacaattggatatgatcctttgttctcaaggtttcttcagcagttcttgctgtatttctctctgcatcagatgtcgttgggtcttttccgcgtaatgggttctttaggccgacatatgatagttgccaatacgtttgggtcctttgcaatgcttgtggtcatgactcttggaggatttataatttccagagatagcataccaagctggtggatatggggttactggatttctccattgatgtatgctcaaaacgcagcttctgtgaatgagttccttggtcataattggcaaaagactgctgggaatcacactagcgactctcttggtttggcattgctgaaagaaagaagtttgttctctgggaactactggtattggatcggtgtcgctgccttgcttggatacacagttctttttaatatactatttacgctgtttttagctcacctcaacccatggggtaagtttcaagccgttgtatccagagaagagttagacgagagagaaaagaaaagaaaaggcgatgagtttgttgtggaactaagagagtacttgcagcattcaggctcaatacatggaaaatatttcaagaatcgaggcatggttctcccatttcaaccactatctctgtctttcagcaatatcaactactatgtggatgttcctttgggactaaaagaacaagggatactagaagataggttgcagctacttgtgaatattactggagcttttagacctggtgtgcttacagcattagtgggagtaagtggtgccggtaaaacaacgctcatggatgttttagctggaagaaaaaccggaggaactatagaaggcgatgtgtacatatctggttttcctaaaagacaggaaacgtttgcaagaatttctggttattgtgagcagaatgatgttcactctccatgcctaactgttgttgaatctctacttttctcggcgtgccttcgtttaccagcagacattgactcagagacacaaagggcgtttgttcatgaggtgatggagctagtggagctgacctctttaagcggggcattagtcggtcttcctggagttgatggcttatcaactgaacagaggaaacggttaacaattgctgtcgagctagttgcaaacccttctatagtattcatggatgagccaacttcaggattggatgcacgggcggccgccattgtgatgaggactgtaagaaatattgttaacacagggcgaaccattgtctgcacgattcatcagcctagcattgatatttttgagtcatttgacgagcttttgttcatgaaacgtggtggagaactcatatatgccggtccacttggccagaagtcttgtgagcttatcaagtatttcgagtcaattgaaggggtgcaaaagatcaaacctggccataatccggcagcatggatgcttgatgtcactgcttctaccgaggaacaccggcttggagttgattttgctgaaatttacaggaactcaaatctttgtcaacgcaacaaggagctgatcgaagtactcagcaagccaagtaacattgcaaaagaaatcgagtttccaaccagatactctcagtcactgtatagtcagtttgttgcttgcctgtggaaacaaaacctatcatattggcgaaacccacaatacactgcagttcgattcttctacaccgtggttatctcgctgatgcttggaacaatatgttggaaatttggctccaaaagggacactcaacaacagttatttaatgcaatgggatcgatgtacgctgcggttctcttcattggaatcaccaatgcaaccgctgcacagcccgttgtttccatagaaagatttgtttcataccgcgagagagcagcgggaatgtactcggcacttccctttgcatttgcacaggtgtttatagaatttccatacgtgttagcacaatccacaatatacagcaccatattctatgcaatggccgcgttcgaatggtccgcggtgaagttcttgtggtacttattcttcatgtacttttcgataatgtacttcaccttctacgggatgatgacgacagcaatcaccccaaaccacaatgtcgcctcgattatagcagcacctttctacatgctctggaatcttttcagtggcttcatgatcccatacaagaggattcctctgtggtggagatggtactactgggcaaacccggtggcttggacactgtacggtctattggtgtctcagtacggtgatgatgaaagatcggtgaagctatcggacggtatccatcaggtgatggtgaaacaactgcttgaggatgtgatgggatataaacatgacttcttaggtgtctcagctattatggttgtcgccttttgcgtcttcttctcccttgtgtttgcattcgccattaaagctttcaatttccagagaagatga

SEQ ID NO: 6 - protein, 1420 aa, Artificial Sequence
  amino acid sequence of AtPDR4 protein
MWNSAENAFSRSTSFKDEIEDEEELRWAALQRLPTYSRIRRGIFRDMVGEPKEIQIGNLEASEQRLLLDRLVNSVENDPEQFFARVRKRFDAVDLKFPKIEVRFQNLMVESFVHVGSRALPTIPNFIINMAEGLLRNIHVIGGKRNKLTILDGISGVIRPSRLTLLLGPPSSGKTTLLLALAGRLGTNLQTSGKITYNGYDLKEIIAPRTSAYVSQQDWHVAEMTVRQTLEFAGRCQGVGFKYDMLLELARREKLAGIVPDEDLDIFMKSLALGGMETSLVVEYVMKILGLDTCADTLVGDEMIKGISGGQKKRLTTGELLVGPARVLFMDEISNGLDSSTTHQIIMYMRHSTHALEGTTVISLLQPSPETYELFDDVILMSEGQIIYQGPRDEVLDFFSSLGFTCPDRKNVADFLQEVTSKKDQQQYWSVPFRPYRYVPPGKFAEAFRSYPTGKKLAKKLEVPFDKRFNHSAALSTSQYGVKKSELLKINFAWQKQLMKQNAFIYVFKFVQLLLVALITMTVFCRTTMHHNTIDDGNIYLGSLYFSMVIILFNGFTEVPMLVAKLPVLYKHRDLHFYPSWAYTLPSWLLSIPTSIIESATWVAVTYYTIGYDPLFSRFLQQFLLYFSLHQMSLGLFRVMGSLGRHMIVANTFGSFAMLVVMTLGGFIISRDSIPSWWIWGYWISPLMYAQNAASVNEFLGHNWQKTAGNHTSDSLGLALLKERSLFSGNYWYWIGVAALLGYTVLFNILFTLFLAHLNPWGKFQAVVSREELDEREKKRKGDEFVVELREYLQHSGSIHGKYFKNRGMVLPFQPLSLSFSNINYYVDVPLGLKEQGILEDRLQLLVNITGAFRPGVLTALVGVSGAGKTTLMDVLAGRKTGGTIEGDVYISGFPKRQETFARISGYCEQNDVHSPCLTVVESLLFSACLRLPADIDSETQRAFVHEVMELVELTSLSGALVGLPGVDGLSTEQRKRLTIAVELVANPSIVFMDEPTSGLDARAAAIVMRTVRNIVNTGRTIVCTIHQPSIDIFESFDELLFMKRGGELIYAGPLGQKSCELIKYFESIEGVQKIKPGHNPAAWMLDVTASTEEHRLGVDFAEIYRNSNLCQRNKELIEVLSKPSNIAKEIEFPTRYSQSLYSQFVACLWKQNLSYWRNPQYTAVRFFYTVVISLMLGTICWKFGSKRDTQQQLFNAMGSMYAAVLFIGITNATAAQPVVSIERFVSYRERAAGMYSALPFAFAQVFIEFPYVLAQSTIYSTIFYAMAAFEWSAVKFLWYLFFMYFSIMYFTFYGMMTTAITPNHNVASIIAAPFYMLWNLFSGFMIPYKRIPLWWRWYYWANPVAWTLYGLLVSQYGDDERSVKLSDGIHQVMVKQLLEDVMGYKHDFLGVSAIMVVAFCVFFSLVFAFAIKAFNFQRR

SEQ ID NO: 7 - DNA, 4362 bp, Artificial Sequence
  nucleotide sequence encoding AtPDR6 protein
atgttaggacgagatgaagatctggtccgaacaatgagcgggagaggaagtttaggctccacaagtcaccggagtttagccggagctgcttcaaaaagcttcagagatgtgtttgctcccccaacggacgacgtgtttggtcggagcgatagacgagaagaagatgacgtggagctccgatgggcggcgcttgagagattaccgacttacgatcggttaagaaaaggtatgttgccgcaaacgatggtcaacggtaagattggacttgaagatgttgacgtcacgaatcttgctcctaaggagaagaaacatcttatggaaatgattctgaaattcgttgaagaagataacgaaaagtttcttcggcgattgagagaaagaactgacagagtgggaatcgaagttccgaaaattgaagtaaggtatgagaatctttcagtggaaggagatgttcgtagtgcaagcagagctcttcctactctcttcaacgtcactttgaatacaatcgagagtattcttggattattccacctgcttccatctaagaagaggaagattgagattcttaaagatataagtggcattatcaaaccatcaaggatgactttattacttggtccacctagttcagggaaaacaactttgttacaagctttagctgggaagcttgatgacactctccagatgtctgggaggataacttactgcggtcatgagtttcgtgagtttgttcctcagaagacttgtgcttacattagtcagcatgaccttcactttggagaaatgactgtgcgagagtcattggacttttcgggacgatgtttaggtgttgggactcggtaccagcttttgactgagctctcaaggagggagagagaagctggaattaagcctgaccctgagattgatgcatttatgaaatccattgctatatctggccaagaaactagtttggttacagactatgtacttaagttacttggtctggacatttgtgctgacacacttgttggagatgtgatgagacgaggtatttctggtggacagcggaaacgtctaacaacaggagagatgttggttggaccagcaacagctcttttcatggatgaaatatcaacagggttagacagttccacaacattccaaatttgcaagttcatgaggcaactagttcatatcgcggatgtcacaatggtcatttcgcttctacaacctgcgccagagacatttgagcttttcgacgacattatcctactctcagagggccaaattgtctaccaggggtcacgagacaacgttcttgagttctttgagtacatgggtttcaaatgtcctgaaaggaaaggtattgcagattttctgcaagaagttacgtctaagaaggaccaagaacagtattggaacaggagagaacatccttacagctatgtatcagtgcatgacttctcaagcggcttcaattcttttcacgcagggcaacaacttgcttcagaattcagggttccttatgacaaagcaaaaactcatcctgcagcactagtgacacaaaagtatggtatatcaaacaaggatctattcaaggcatgctttgatagagaatggcttcttatgaaacgtaactcctttgtgtacgtgttcaagaccgtccagataaccatcatgtctttgattgccatgacggtttattttcggacagaaatgcatgtcggcactgtgcaagatggtcaaaagttttatggtgctctctttttcagcttgatcaatctaatgtttaatggaatggctgaactagccttcacagtgatgaggcttccggttttcttcaagcagagggacttcttgttctatcctccatgggctttcgcattacccggttttcttctcaagattccgttatctcttattgaatcagttatatggatcgctcttacatactacactattggttttgctccttctgctgccaggttcttccggcagttgctagcatacttctgtgtgaatcagatggcactttctttatttagattccttggagcccttggaagaacagaagtcatcgccaactctggcgggacattagcattgctagtcgtatttgttcttggaggctttattatttctaaagatgacatcccgtcgtggctgacttggtgctattatacatcacctatgatgtatggacagactgcattagttataaatgaatttttggatgagcgatggggcagcccaaacaatgatacacgcatcaacgcgaaaacagttggagaagtcttgctgaagagcagaggcttctttacagaaccatattggttttggatctgtattggggcgctacttgggtttactgtgttgttcaacttctgttacataatagccttgatgtatttgaaccctcttggtaactccaaagctactacagttgtggaagaaggtaaagacaaacacaaagggagtcacagtggaacaggaggttctgtagtggaactcactagtacttcaagtcatggaccaaagaaaggaatggttttacctttccaaccactttctctagcattcaacaatgtgaactactacgtggatatgcctgcagaaatgaaggctcaaggagttgaaggagatcgacttcagttactaagagacgttggtggagctttcaggcctggcgtattgacagcattggtcggtgttagtggtgcgggtaagactaccttaatggatgtcttggccggtaggaaaacaggaggctatgtagaagggagtattaacatatctggttacccaaagaaccaagcaacatttgctagagtcagtggttactgtgagcaaaatgatatccattctccacatgttaccgtttatgaatcccttatctattcagcatggcttcgtctttccgccgatatagataccaaaacacgagagatgtttgtggaggaagtgatggagttggtggagcttaaacctcttagaaactctatagttggtcttcctggtgtagatggtctttcaacagaacagaggaagaggcttactatagcagttgaattggtagctaatccatcaataatcttcatggacgagccaacatctggtcttgatgcaagagctgctgccattgttatgcgtactgttaggaatactgttgatacaggaagaactgttgtttgtacaattcaccaacctagcatcgacattttcgaatccttcgatgagttgctgttgatgaaacgaggaggacaagttatatatgctggaacactaggacatcactcacaaaaactcgttgaatactttgaggctattgaaggggttccaaagatcaaggacggatacaatcctgcgacgtggatgcttgacgtcactactccttcaatggagtcacaaatgagcgtggactttgctcaaatattcgttaactcctctgttaatcggagaaatcaggaactcatcaaagagctgagtactccaccaccgggatcgaatgatctctacttccgaaccaagtacgcgcaaccattttctactcaaaccaaagcttgcttctggaaaatgtattggtcaaactggagatatcctcagtacaatgccattcggtttctcatgacagtagtcattggtgtcttgtttggtctacttttctggcaaacaggaactaaaatagagaaagagcaagacctgaataatttctttggagccatgtatgctgctgtattgttcctcggtgccaccaacgctgcaacagttcaacccgcagttgctattgagcgaacggttttctaccgcgaaaaagccgctggaatgtactccgccattccctatgcaatttctcaggtagcagtagaaatcatgtacaacactatacaaaccggagtttacacacttatcctttactcgatgatcggatacgactggactgtggtcaaattcttctggttctactactacatgttaacatgcttcgtctacttcaccctatacggtatgatgcttgtcgccttgacaccaaactatcaaatagctggaatctgcttgtccttcttcctcagtttctggaatcttttctccggtttcctcatccctagaccgcaaataccaatatggtggagatggtactattgggcatcacctgtggcttggacattgtatggaatcatcacatctcaagtaggagacagggattcgattgtgcacatcactggtgttggagatatgagtcttaaaacgttgctcaaaaatggattcgggttcgattatgacttcttaccagttgtagccgttgtccacatcgcctggatcttgatctttctctttgcctttgcttatggtatcaagttcctcaacttccaaagaaggtga

SEQ ID NO: 8 - protein, 1453 aa, Artificial Sequence
  amio acid sequence of AtPDR6 protein
MLGRDEDLVRTMSGRGSLGSTSHRSLAGAASKSFRDVFAPPTDDVFGRSDRREEDDVELRWAALERLPTYDRLRKGMLPQTMVNGKIGLEDVDVTNLAPKEKKHLMEMILKFVEEDNEKFLRRLRERTDRVGIEVPKIEVRYENLSVEGDVRSASRALPTLFNVTLNTIESILGLFHLLPSKKRKIEILKDISGIIKPSRMTLLLGPPSSGKTTLLQALAGKLDDTLQMSGRITYCGHEFREFVPQKTCAYISQHDLHFGEMTVRESLDFSGRCLGVGTRYQLLTELSRREREAGIKPDPEIDAFMKSIAISGQETSLVTDYVLKLLGLDICADTLVGDVMRRGISGGQRKRLTTGEMLVGPATALFMDEISTGLDSSTTFQICKFMRQLVHIADVTMVISLLQPAPETFELFDDIILLSEGQIVYQGSRDNVLEFFEYMGFKCPERKGIADFLQEVTSKKDQEQYWNRREHPYSYVSVHDFSSGFNSFHAGQQLASEFRVPYDKAKTHPAALVTQKYGISNKDLFKACFDREWLLMKRNSFVYVFKTVQITIMSLIAMTVYFRTEMHVGTVQDGQKFYGALFFSLINLMFNGMAELAFTVMRLPVFFKQRDFLFYPPWAFALPGFLLKIPLSLIESVIWIALTYYTIGFAPSAARFFRQLLAYFCVNQMALSLFRFLGALGRTEVIANSGGTLALLVVFVLGGFIISKDDIPSWLTWCYYTSPMMYGQTALVINEFLDERWGSPNNDTRINAKTVGEVLLKSRGFFTEPYWFWICIGALLGFTVLFNFCYIIALMYLNPLGNSKATTVVEEGKDKHKGSHSGTGGSVVELTSTSSHGPKKGMVLPFQPLSLAFNNVNYYVDMPAEMKAQGVEGDRLQLLRDVGGAFRPGVLTALVGVSGAGKTTLMDVLAGRKTGGYVEGSINISGYPKNQATFARVSGYCEQNDIHSPHVTVYESLIYSAWLRLSADIDTKTREMFVEEVMELVELKPLRNSIVGLPGVDGLSTEQRKRLTIAVELVANPSIIFMDEPTSGLDARAAAIVMRTVRNTVDTGRTVVCTIHQPSIDIFESFDELLLMKRGGQVIYAGTLGHHSQKLVEYFEAIEGVPKIKDGYNPATWMLDVTTPSMESQMSVDFAQIFVNSSVNRRNQELIKELSTPPPGSNDLYFRTKYAQPFSTQTKACFWKMYWSNWRYPQYNAIRFLMTVVIGVLFGLLFWQTGTKIEKEQDLNNFFGAMYAAVLFLGATNAATVQPAVAIERTVFYREKAAGMYSAIPYAISQVAVEIMYNTIQTGVYTLILYSMIGYDWTVVKFFWFYYYMLTCFVYFTLYGMMLVALTPNYQIAGICLSFFLSFWNLFSGFLIPRPQIPIWWRWYYWASPVAWTLYGIITSQVGDRDSIVHITGVGDMSLKTLLKNGFGFDYDFLPVVAVVHIAWILIFLFAFAYGIKFLNFQRR

SEQ ID NO: 9 - DNA, 4221 bp, Artificial Sequence
  nucleotide sequence encoding AtPDR10 protein
atggcacattacagagtaagctcagaggtagagaacataatgaacagagacaggagtcaccgaaaaaacgaagaagaagacgaagaggaagctttgaaattagcggctatggagaagttgcagcgtcttccaacgtatgaccgggcgagaaaggccgtgttaaaagggatcacaggaggtttcaaggagattgacatgaaagaccttggcctcgcggagagaagagagttattcgatagagttatgacgatggatgatgaagattggcatggagagtatctaaggaggctaaagagtcgttttgatagagtctctctgcatctgccgacgatagaagtccgatttgaagatttaaacgtgactgccgaagcttatgcgggaagcaaaaccgttcccacagtcctgaactcatacgtcaaccttttaaaaggtattggaactaaaatcagagttcttccggatcggaaaaagagaatctccattcttaacgatgttagtggaatcatcaagcctggcagattgactctactattgggaccaccgggctctggaaaatcgacactactaaaagcattgtctggaaagacagaaactggactaagatctacagggaaagtgacatacaatggtcatgaactgcatgagtttgtgcctgaaagaactgcgggatacatcgaccaatatgatgttcacttgccggacctaacagttcgagaaacattaaagttttccgcaaaatgccaaggagttggcacaggctacgatatgcttgctgagttactgagacgagagaaagacttgaatatcaagccggatccttacctcgatgcgttaatgaaggcatcggttataaaaggacacaaggagtatgtggttacagattacgttttaaaagtcttaggacttgagatatgtgctgatacgatagttgggaatcatatgaagagaggcatctctggtgggcaaaagaaacgcgtaacgactggcgaaatgttggtcggtccagttggagctttcttcatggacaacatatcagatggtttagatagctcgacgacgtttcagattgtcaagagcatcaagcaaatgatccatgttttcgataaaaccgctcttatttctcttctccagcctcctccagagacatttgagctcttcgatgatgtcatcattcttggagaaggccacattgtttaccaaggccctcgagaagacgtcctcgagttttttgaatttatggggtttaagtgtcctgaaagaaaagggattgctgattacttgcaagaaattttatccaaaaaagatcaagaacaatattgggctaatccagagctgccatatcgttatgtaaccgcaaaaaaattcgaggaaggcttcaaaattcatcatttcgggagagcaatgcggtctcagcttgcaacgccatttgatcgactaaagaatcacagagcagccctgacaaggacaacatatggagctagcaagttggaactactaaaggcatgcttagaacgagaaagcattctaatgaagaggaacttacgcactttcgtcttgaagtcacttcagctaattatcaatgccattctaatcggagttgtgttttggcaacaaaagaactatcccagcacggtcgaggatggaatcatctacatgggagctatctacttggaagtgcaaatgattgtgttctcggggtttttcgaacttccaatgactatcgataagcttccagtgttctacaagcaacgtcattttagcttttacccgtcatgggcattctcgttaccaacgtcaatcatcaccttccctttatccttcgttgaagtcttcattgtggtcttaataacttatttcaccattggatatgatctaaccgtcccatcgttcctgaaacattacttagttctagcattgtgcggacaaatgtcgtatgggctttttcgatgtatcgctgcagtgactcgaaatcatgtggtttcaaacacaatgggatgtcttgctgtgatgtggctgatgacattcagcggatatgtgttgtcccgaaatcaagtgcataaatggttgacatgggcgtactggacatcaccaatgatgtatatccaaacagctgtttcggttaacgaattccgcagcgagtcttggaaagatgtaatttctaagaaacctttctttaagttttcaacgtcacattttaaagatatcaaacttaaccgtgtagtgtacgatttccagggtctaggagtagcggtcttgaaatcgcgagaatatggaatatctaagactgcggtgttaccggatgaacgagaagaagctgatagcaacaatacgaccggcagagactatacaggaactacgatggaacgattctttgatagagttgtaacaacaagaacttgcaacgacaaaaaactgcgtatacctttcaaacctctatatatgacgttcgagaacatcacatattctgtagacaccccaaaggaaatgaaagaaaaaggcataagagagaataaactagtactattgaatggattaagcggtgcttttaggcccggggttcttacagcacttatgggcgtgagtggtgcgggcaaaactactttgatggatgtcttagccggacgtaagaacacaggatacatccaaggggaaatatatgtctctggatttcctaaaaaacaggatagctttgctcgtgtttcgggttattgcgaacaatctgacatccattcccctcttttaactgtttatgagtctcttctttattctgcatggttgaggttgcctcctgatatcgacacacatactcgagaggtgatggaattgatagagttaaaagcattgagggagatgttggtaggatatgtgggaattagtgggctttcgaccgagcaaagaaaaagaatgacaatcgcagtagagcttgtagccaatccttctattcttttcatggatgagcctacatccggcttggatgctagagctgccgcgattgtcatgaggacagttcgaaacactgttgacactggaagaactgttgtatgcaccattcaccagccaagtattgatatttttgaatcctttgatgagcttttcttgttgacaagaggaggcgaagagatctatgttggtccaataggccaccattcctctcaactaatcgaatactttgagggaataagaggagtaggaaaaataaaggaaggttataatccagcaacatgggcccttgaagtaacaacaagggcacaagaagatgttcttggtgtcacatttgcgcaagtatacaagaagtcaaacctctataggagaaacaaagatctgattaaggaactgaacaatattcctcctcatgcacaagacattcatttctcaacaaaatattcacaatcctacctctctcaattccaagcttgcttgtggaagcaacacaaatcatattggagaaatgttccttataacgcggtgcgtttcagtttcggggctgctgttggaatcatgtatggaatcatcttttggagcctcggaaaacgaaaaggaacaaggcaagatatcttcaatagtgtaggcgcgatgtcaaccgttgttggcttcttaagttcacaaagtgcggctactgtacgtccagttgtaatcgctgaacgtactgttttttatcgagaagccggtgctggaatgtactccgctctaccctatgcattttcacaggtgataatcgagatcccatatacaatggctcaagcttgcatctatggagttattgtctatgggatgataggatacgaatggacagcttccaagttttttttgaacatattctttaccttcatcagcatcctctactctatatacactggtattatggtcatttccgtaagccctaatcaagaaatcgcttcgatactcaatggtgtcatatccacatcgtggaacgtcttttcaggattcactattcctcgtcccagaatgcatgtatggttgagatggttcacctacgtgtgtcccgggtggtgggggttgtacggactgacgatagctcaatacggagatgtggagacaaggctcgatacgggtgagacggttgttgagttcatgaagaattattatggttatgaatacaacttcttgtgggttgtctcacttaccctcattgctttctctatgttttttgtatttatctatgctttctctgttaagattctgaatttccaaaagaggtga

SEQ ID NO: 10 - protein, 1406 aa, Artificial Sequence
  amino acid sequence of AtPDR10 protein
MAHYRVSSEVENIMNRDRSHRKNEEEDEEEALKLAAMEKLQRLPTYDRARKAVLKGITGGFKEIDMKDLGLAERRELFDRVMTMDDEDWHGEYLRRLKSRFDRVSLHLPTIEVRFEDLNVTAEAYAGSKTVPTVLNSYVNLLKGIGTKIRVLPDRKKRISILNDVSGIIKPGRLTLLLGPPGSGKSTLLKALSGKTETGLRSTGKVTYNGHELHEFVPERTAGYIDQYDVHLPDLTVRETLKFSAKCQGVGTGYDMLAELLRREKDLNIKPDPYLDALMKASVIKGHKEYVVTDYVLKVLGLEICADTIVGNHMKRGISGGQKKRVTTGEMLVGPVGAFFMDNISDGLDSSTTFQIVKSIKQMIHVFDKTALISLLQPPPETFELFDDVIILGEGHIVYQGPREDVLEFFEFMGFKCPERKGIADYLQEILSKKDQEQYWANPELPYRYVTAKKFEEGFKIHHFGRAMRSQLATPFDRLKNHRAALTRTTYGASKLELLKACLERESILMKRNLRTFVLKSLQLIINAILIGVVFWQQKNYPSTVEDGIIYMGAIYLEVQMIVFSGFFELPMTIDKLPVFYKQRHFSFYPSWAFSLPTSIITFPLSFVEVFIVVLITYFTIGYDLTVPSFLKHYLVLALCGQMSYGLFRCIAAVTRNHVVSNTMGCLAVMWLMTFSGYVLSRNQVHKWLTWAYWTSPMMYIQTAVSVNEFRSESWKDVISKKPFFKFSTSHFKDIKLNRVVYDFQGLGVAVLKSREYGISKTAVLPDEREEADSNNTTGRDYTGTTMERFFDRVVTTRTCNDKKLRIPFKPLYMTFENITYSVDTPKEMKEKGIRENKLVLLNGLSGAFRPGVLTALMGVSGAGKTTLMDVLAGRKNTGYIQGEIYVSGFPKKQDSFARVSGYCEQSDIHSPLLTVYESLLYSAWLRLPPDIDTHTREVMELIELKALREMLVGYVGISGLSTEQRKRMTIAVELVANPSILFMDEPTSGLDARAAAIVMRTVRNTVDTGRTVVCTIHQPSIDIFESFDELFLLTRGGEEIYVGPIGHHSSQLIEYFEGIRGVGKIKEGYNPATWALEVTTRAQEDVLGVTFAQVYKKSNLYRRNKDLIKELNNIPPHAQDIHFSTKYSQSYLSQFQACLWKQHKSYWRNVPYNAVRFSFGAAVGIMYGIIFWSLGKRKGTRQDIFNSVGAMSTVVGFLSSQSAATVRPVVIAERTVFYREAGAGMYSALPYAFSQVIIEIPYTMAQACIYGVIVYGMIGYEWTASKFFLNIFFTFISILYSIYTGIMVISVSPNQEIASILNGVISTSWNVFSGFTIPRPRMHVWLRWFTYVCPGWWGLYGLTIAQYGDVETRLDTGETVVEFMKNYYGYEYNFLWVVSLTLIAFSMFFVFIYAFSVKILNFQKR

SEQ ID NO: 11 - DNA, 4173 bp, Artificial Sequence
  nucleotide sequence encoding AtPDR13 protein
atggctcaaacaggtgaagatgttgacaaggcaaagtccttccaagttgagtttgcttgtggaaatggtgttgatgatgaggagaagcttcggtcgcagtgggcaacagttgagagattaccaacttttaaaagggttactactgctttgttacacacaggagatgactcgtcagatattattgatgttactaaacttgaggatgctgagagacggttgttgattgaaaagctcgtcaaacaaatcgaagctgataacctccgtttactcaggaaaataagaaagagaatcgacgaagttggtatagagttaccaacggtggaagtgaggttcaatgacctctctgttgaagcggaatgccaagtagttcatggaaagcctatcccaactctttggaatactatcaagggctcactatctaagttcgtttgttcaaagaaagaaaccaagataggcatcttgaaaggagtgagtggaattgtaaggcctggaagaatgacattgttgcttggtcctcctggttgtggtaaaaccactcttctacaagcactttctggaagactttcccattctgtaaaggttggaggaaaagtaagttataatggttgcttactttcagagtttattccagaaaaaacgtcaagttatatcagtcaaaatgatctacacattccagagctgagtgtgagagagactctcgacttctctgcgtgttgccaaggcataggaagccgtatggaaattatgaaagagatcagtagaagggagaaactaaaagaaattgttccagatcctgatatagatgcttacatgaaggcaatatctgttgaaggtttgaaaaacagtatgcaaactgactatattctaaagatcctgggactcgatatctgtgcagatacacgagcaggagatgccacaagacctggaatatctggtggccaaaagaggaggttgacaacagctactactctgttaatggatgaaatatcgaatggtttggacagctcaactacgttccagatagtgtcgtgcctccaacagttggcacatatagctggagccaccatactgatttcacttcttcagcctgcaccagaaacatttgagctttttgacgatgtgattcttttgggggaaggaaagataatttaccatgctccaagagctgatatctgtaaattctttgaaggttgtggatttaaatgtccagagagaaaaggcgttgctgacttcctccaggaggttatgtctagaaaagatcaagaacaatattggtgccacagaagcaagccctacagttatatatctgttgattcattcattaagaagtttaacgaatcaaatcttgggtttttgctgaaagaagaactgtcaaagccgtttgataaatcccagactcgcaaggatagtctatgttttagaaaatactcactcagtaaatgggagatgcttaaagcttgctcaaggagagaaattcttttgatgaaacggaattctttcatttacttgttcaaatctggactgttagtgttcaatgcgttagtcacaatgaccgtttttctacaagctggagctacgagggatgctcgtcatgggaattatcttatgggttctatgttcactgctctctttagacttcttgccgatgggcttccagaactcactttgactatctccagattgggagtgttctgcaaacagaaagatttatacttctatcctgcttgggcatatgcaattccttcgattatcttaaggatacctctttcggttcttgattcatttatttggacagtactgacatattatgtcattggttacagtcccgaagtcggaaggttcttccggcacttcattatcttacttacattccacctttcatgtatatcaatgtttcgcgctatagcgtcaatctgtcgcacatttgttgcttgctcaattactggagctatttcggtattgcttctcgcattgtttggaggctttgtgattccaaaatcatccatgcctacttggctgggttggggattctggctttctcccttgtcatatgctgagattggtttaaccgcaaatgaatttttctctccacggtggagaaagttaacctctggcaacattacagccggggaacaagtgttagatgtccgtggattgaattttggtaggcactcttactggacagcttttggtgctttagttgggtttgtcttgttcttcaatgccctctacacgttggccctgacatatcggaataatccacaaagatcccgtgctattgtctcgcatgggaagaactctcagtgctcagaagaagattttaaaccctgtcctgaaatcacatcgcgagctaaaacaggaaaagttatcttgccttttaagccactcactgtcacatttcaaaacgtccagtattatattgagactcctcaggggaagacacggcaacttctctttgatattacaggcgcgttgaagcccggtgttctcacatctctgatgggtgtcagtggagcagggaaaacgactcttcttgatgtcctttctggaaggaaaacccgcggtatcatcaaaggagagatcagagtaggcgggtatcctaaggttcaagaaacatttgcacgagtatcaggttactgtgaacagtttgatattcattcccctaatataactgtggaagagtctttgaaatattctgcttggcttcgactcccctataacatcgatgcaaagaccaagaacgaactcgtcaaagaagtcctcgagacagttgagcttgaggatatcaaagattccatggtgggacttcctggaataagtggtttatctacagaacaacgcaaaaggctgacaatagccgtggaacttgtttctaacccttccatcatatttctggatgaacctacaacagggctggatgcaagagccgcagccattgttatgagagctgtgaaaaatgttgctgagactgggagaacagttgtttgcacgatccaccagcctagcatagatatctttgagacatttgatgagctgatcctgatgaaagatgggggacagcttgtctactatggtcctcttggaaaacattcaagtaaggttatcaaatactttgagagcatccctggagttccaaaagtccaaaagaattgtaatccagccacttggatgttagatattacttgtaaatctgcagagcacagacttggaatggattttgcacaagcatacaaggattcaactctgtacaaggagaacaaaatggtggttgaacaactgagttctgcgtctctgggatcagaagctctgagctttccttcacgttattcacaaacaggttgggggcaactaaaggcttgcctttggaaacaacattgctcgtattggagaaacccttcacataatctcactcgcatagtcttcatattactcaattctctgttatgtagccttctcttctggcaaaaagctaaggacataaataatcagcaagatctttttagcatatttggctcaatgtacactatagtaatcttctcgggaataaacaactgtgcgacagttatgaacttcattgcaaccgagcgcaatgttttctaccgtgaaaggtttgcgcggatgtactcctcatgggcgtattcattttctcaggtcctagttgaggttccatactcactactccagtctctactatgtacgatcattgtatatcctatgatcggctaccatatgtctgtttacaagatgttttggagcttgtacagcatcttctgctcgttgcttatcttcaactactgtgggatgcttatggttgctttgacgccaaacattcacatggcattgactttgcgctcaacttttttctccatggtgaatctgtttgctggctttgtcatgccaaaacagaaaatcccaaaatggtggatatggatgtactacctgagccctacatcgtgggtgttggaaggattactgagttcgcagtatggagatgtcgagaaagagataacagtatttggagagaagaagagtgtttcagctttcttggaggattacttcggctacaaacatgactccttggctgttgtagcgtttgtcctcattgcttttcctatcatcgttgcttctctttttgccttcttcatgagcaaactcaattttcaaaagaaatag

SEQ ID NO: 12 - protein, 1390 aa, Artificial Sequence
  amino acid sequence of AtPDR13
MAQTGEDVDKAKSFQVEFACGNGVDDEEKLRSQWATVERLPTFKRVTTALLHTGDDSSDIIDVTKLEDAERRLLIEKLVKQIEADNLRLLRKIRKRIDEVGIELPTVEVRFNDLSVEAECQVVHGKPIPTLWNTIKGSLSKFVCSKKETKIGILKGVSGIVRPGRMTLLLGPPGCGKTTLLQALSGRLSHSVKVGGKVSYNGCLLSEFIPEKTSSYISQNDLHIPELSVRETLDFSACCQGIGSRMEIMKEISRREKLKEIVPDPDIDAYMKAISVEGLKNSMQTDYILKILGLDICADTRAGDATRPGISGGQKRRLTTATTLLMDEISNGLDSSTTFQIVSCLQQLAHIAGATILISLLQPAPETFELFDDVILLGEGKIIYHAPRADICKFFEGCGFKCPERKGVADFLQEVMSRKDQEQYWCHRSKPYSYISVDSFIKKFNESNLGFLLKEELSKPFDKSQTRKDSLCFRKYSLSKWEMLKACSRREILLMKRNSFIYLFKSGLLVFNALVTMTVFLQAGATRDARHGNYLMGSMFTALFRLLADGLPELTLTISRLGVFCKQKDLYFYPAWAYAIPSIILRIPLSVLDSFIWTVLTYYVIGYSPEVGRFFRHFIILLTFHLSCISMFRAIASICRTFVACSITGAISVLLLALFGGFVIPKSSMPTWLGWGFWLSPLSYAEIGLTANEFFSPRWRKLTSGNITAGEQVLDVRGLNFGRHSYWTAFGALVGFVLFFNALYTLALTYRNNPQRSRAIVSHGKNSQCSEEDFKPCPEITSRAKTGKVILPFKPLTVTFQNVQYYIETPQGKTRQLLFDITGALKPGVLTSLMGVSGAGKTTLLDVLSGRKTRGIIKGEIRVGGYPKVQETFARVSGYCEQFDIHSPNITVEESLKYSAWLRLPYNIDAKTKNELVKEVLETVELEDIKDSMVGLPGISGLSTEQRKRLTIAVELVSNPSIIFLDEPTTGLDARAAAIVMRAVKNVAETGRTVVCTIHQPSIDIFETFDELILMKDGGQLVYYGPLGKHSSKVIKYFESIPGVPKVQKNCNPATWMLDITCKSAEHRLGMDFAQAYKDSTLYKENKMVVEQLSSASLGSEALSFPSRYSQTGWGQLKACLWKQHCSYWRNPSHNLTRIVFILLNSLLCSLLFWQKAKDINNQQDLFSIFGSMYTIVIFSGINNCATVMNFIATERNVFYRERFARMYSSWAYSFSQVLVEVPYSLLQSLLCTIIVYPMIGYHMSVYKMFWSLYSIFCSLLIFNYCGMLMVALTPNIHMALTLRSTFFSMVNLFAGFVMPKQKIPKWWIWMYYLSPTSWVLEGLLSSQYGDVEKEITVFGEKKSVSAFLEDYFGYKHDSLAVVAFVLIAFPIIVASLFAFFMSKLNFQKK

SEQ ID NO: 13 - DNA, 305 bp, Artificial Sequence
  a part of AtPDR12 gene for RNAi
gcaaatccttccatcatattcatggatgaacctacttcaggattggatgcacgagctgctgccatcgttatgaggactgtaaggaacacagttgacactggtagaacagtcgtctgcaccattcaccagcctagcatcgacatctttgaagcctttgatgagttgttcctacttaagcgtggaggtgaggagatatacgttggacctcttggccacgaatcaacccatttgatcaactattttgagagtattcaaggaatcaacaagatcacagaaggatacaacccagcaacctggatgcttga

SEQ ID NO: 14 - DNA, 28 bp, Artificial Sequence
  restriction site for HindIII
aagcttacgccggccgccgccgcggcag

SEQ ID NO: 15 - DNA, 28 bp, Artificial Sequence
  restriction site for BamHI
ggatcctttgtatccaagaaatcaaagt

SEQ ID NO: 16 - DNA, 45 bp, Artificial Sequence
  AtPDR12-TFF primer
cccgggggggatccatggagggaactagttttcaccaagcgagta

SEQ ID NO: 17 - DNA, 46 bp, Artificial Sequence
  AtPDR12-TFR primer
ggatccgcggccgcctatcgtttttggaaattgaaactcttgattc

SEQ ID NO: 18 - DNA, 62 bp, Artificial Sequence
  Ri-F primer
ggggacaagtttgtacaaaaaagcaggcttcatggcaaacccttctatagtattcatggatg

SEQ ID NO: 19 - DNA, 60 bp, Artificial Sequence
  Ri-R primer
ggggaccactttgtacaagaaagctgggtcttaatcaagcatccatgctgccggattatg

SEQ ID NO: 20 - DNA, 18 bp, Artificial Sequence
  oligo dT primer
tttttttttttttttttt

SEQ ID NO: 21 - DNA, 24 bp, Artificial Sequence
  P123rd-F primer
ctgcttttgggtcctccaagttct

SEQ ID NO: 22 - DNA, 23 bp, Artificial Sequence
  P123rd-R primer
gagattgaatgtctctggcgcag

SEQ ID NO: 23 - DNA, 20 bp, Artificial Sequence
  Tub-F primer
gctgacgttttctgtattcc

SEQ ID NO: 24 - DNA, 20 bp, Artificial Sequence
  Tub-R primer
aggctctgtattgctgtgat

SEQ ID NO: 25 - DNA, 24 bp, Artificial Sequence
  AtPDR4-F primer
gcattagtgggagtaagtggtgcc

SEQ ID NO: 26 - DNA, 23 bp, Artificial Sequence
  AtPDR4-R primer
ttgagtgtcccttttggagccaa

SEQ ID NO: 27 - DNA, 24 bp, Artificial Sequence
  AtPDR10-F primer
gaatggattaagcggtgcttttag

SEQ ID NO: 28 - DNA, 24 bp, Artificial Sequence
  AtPDR10-R primer
ttgacatcgcgcctacactattga

SEQ ID NO: 29 - DNA, 24 bp, Artificial Sequence
  AtPDR12-F primer
ctgcttttgggtcctccaagttct

SEQ ID NO: 30 - DNA, 23 bp, Artificial Sequence
  AtPDR12-R primer
gagattgaatgtctctggcgcag

SEQ ID NO: 31 - DNA, 20 bp, Artificial Sequence
  LP primer
gccaagatccaagacaaaga

SEQ ID NO: 32 - DNA, 20 bp, Artificial Sequence
  RP primer
ttacgagcaagcatcatcaa

(19) United States
(12) Patent Application Publication
(10) Pub. No.: US 2012/0042410 A1
(43) Pub. Date: Feb. 16, 2012

(54) ABSCISIC ACID CARRIER GENE AND TRANSGENIC PLANT EXPRESSING THE SAME

(75) Inventors: Young-Sook Lee, Gyeongsangbuk-do (KR); Mi-Young Lee, Gyeongsangbuk-do (KR); Enrico Martionia, Zurich (CH); Joo-Hyun Kang, Gyeongsangbuk-do (KR)

(73) Assignee: POSTECH ACADEMY-INDUSTRY FOUNDATION, Pohang-si, Gyeongsangbuk-do (KR)

(21) Appl. No.: 13/144,793

(22) PCT Filed: Jan. 22, 2010
(86) PCT No.: PCT/US2010/000424

§ 371 Date: Jul. 15, 2011

Related U.S. Application Data
(60) Provisional application No. 61/146,705, filed on Jan. 23, 2009.

(51) Int. Cl. C12N 15/82; A01H 5/00; C07H 21/04; C12N 5/10; C07K 14/00

(52) U.S. Cl. 800/278; 435/419; 435/320.1; 530/350; 536/23.1; 800/298; 800/305; 800/306; 800/307; 800/317.2; 800/317.3; 800/317.4; 800/322

(57) ABSTRACT

The present invention relates to carrier genes of abscisic acid and the transgenic plant prepared using such genes, and more specifically to genes that enhance resistance to salt or dryness in relation to abscisic acid transport and the recombinant vectors including the genes, the transgenic plant prepared using the recombinant vector, a transgenic plant that is superior in terms of resistance to base and dryness, a method for environmental purification in arid regions based on the plants, and the development of crops having enhanced yields.

### TECHNICAL FIELD

The present invention relates to an abscisic acid (ABA) carrier gene and a transgenic plant using the same. More particularly, the present invention relates to a gene which is involved in ABA carrier and which improves resistance to salinity and drought; a recombinant vector carrying the gene; a transgenic plant prepared using the recombinant vector; a transgenic plant modified to be improved in resistance to salinity and/or drought; and a method for environmental remediation in highly salty and/or arid regions and for increasing crop productivity in highly salty and/or arid regions using this transgenic plants.

### BACKGROUND ART

Over the world, water stressed countries have been proliferating, with the concomitant desertification of some arid regions, which has been incurring very severe agricultural and environmental problems. Problems may be found even in regions with sufficient amounts of water because of salts. When agricultural plants are cultivated using water for irrigation, salts which have a negative influence on the growth of crops accumulate in the land because salts contained in the water used for irrigation are left behind upon the evaporation of water. There is thus an urgent need for the development of plants that can grow even in arid regions or under high salt concentrations. Such techniques would make a great contribution to agricultural productivity. Plants that are resistant to both contaminants and drought are ideal for the economical environmental remediation of arid regions. For example, plants which can lower transpirational water loss are advantageous in terms of survival under arid conditions, thus contributing to improvements in agricultural productivity as well as the environmental remediation of arid regions.

ABA is a ubiquitous plant hormone that is accumulated in response to various stress caused by environment including drought, salinity, cold, heat, and infection by pathogens and is involved in a large number of physiological processes including the impartment of plants with stress resistance. When pretreated with ABA, plants can resist stress better, compared to a control. Mutant plants which cannot synthesize or respond to ABA are vulnerable to environmental stress (Taiz and Zeiger, Plant Physiology, 3^{rd} edition: 543˜555, 591˜621). Therefore, the use of the proteins which are involved directly or indirectly in the synthesis, transportation, and responses involving ABA may lead to the development of plants that are improved in their resistance to stresses such as including drought, salinity, cold, heat and infection by pathogens. For example, a plant which rapidly synthesizes ABA in response to stress might be very effective in improving the yield of a crop. In many cases, various types of stress may coexist in the regions which need environmental remediation, thus, the genes implicated in the synthesis, transportation and responses of ABA which is responsible for the protection of plants from various environmental stress are very useful for the development of plants.

The genes implicated in resistance to salinity and drought may also be used to rehabilitate the environment. Environmental rehabilitation is to rehabilitate an ecological system which has been naturally or artificially destroyed, thereby constructing a clean environment, preserving natural resources and setting up a base where human beings can live together with other biological species. Typically, rehabilitation may be achieved by removing pollutants, planting resistant plants, and reintroducing extinct or endangered animals. Salt-resistant genes may be used to rehabilitate agriculture and the environment of reclaimed land because these areas are high in salinity.

Therefore, there is a need for plants resistant to salinity and/or drought.

### DISCLOSURE

### Technical Problem

Accordingly, the present invention has been made keeping in mind the above problems occurring in the prior art, and an object of the present invention is to provide a gene which is involved in ABA transport or which allows the resistance to salinity and/or drought of plants to be improved.

It is another object of the present invention to provide a recombinant vector carrying a gene which is involved in ABA transport or which allows the resistance to salinity and/or drought of plants to be improved.

It is a further object of the present invention to provide a transgenic plant which is involved in ABA transport or which allows the resistance to salinity and/or drought to be improved.

It is still a further object of the present invention to provide a method for generating a transgenic plant which is involved in ABA transport or which allows resistance to salinity and/or drought to be improved.

It is still another object of the present invention to provide a technique for making salty regions and/or arid regions environmentally friendly.

### Technical Solution

Leading to the present invention, the present inventors found that a sequence encoding an ATP-binding cassette (ABC) carriers consisting of two repeats of [six transmembrane domains and one ATP-binding domain] is able to transport ABA whereby resistance to salinity and/or drought can be conferred on the plant expressing the protein.

Therefore, the present invention is characterized in that a sequence encoding an ABC carriers consisting of two repeats of six transmembrane domains and one ATP-binding domain is expressed, overexpressed or down-regulated in plants, thereby improving the ABA transportation and/or resistance to salinity and/or drought of plants.

In accordance with an aspect thereof, the present invention provides the use of an ABC carrier consisting of two repeats of six transmembrane domains and one ATP-binding domain and/or a nucleotide sequence encoding the same in improving ABA transportation and/or resistance to salinity and/or drought. In another aspect thereof, the present invention provides a composition for improving ABA transportation and/or resistance to salinity and/or drought, comprising an ABC carrier consisting of two repeats of six transmembrane domains and one ATP-binding domain and/or a nucleotide sequence encoding the same.

In accordance with a further aspect thereof, the present invention provides a recombinant vector comprising a nucleotide sequence encoding an ABC carrier consisting of two repeats of six transmembrane domains and one ATP-binding domain or a nucleotide sequence sharing a homology therewith; and

a promoter, which can be expressed in plant cells, operably linked to the nucleotide sequence.

In accordance with still a further aspect thereof, the present invention provides a transgenic plant which is transformed with the recombinant vector.

In accordance with still another aspect thereof, the present invention provides a transgenic plant, plant cell, and/or part of a plant, transformed with the recombinant vector.

In accordance with yet another aspect thereof, the present invention provides a method for generating a plant whose capability of transporting ABA is improved, comprising:

(a) constructing a recombinant vector in which a nucleotide sequence coding for an ABA carrier consisting of two repeats of six transmembrane domains and one ATP-binding domain, and a nucleotide sequence having a homology therewith is operably linked to a promoter; and

(b) introducing the recombinant vector into a plant cell or a plant tissue.

The plant improved in ability to transport ABA enjoys the advantage of being further resistant to drought, salinity, cold, heat and infection by pathogens.

In accordance with yet a further aspect thereof, the present invention provides a method for generating a salt-and/or drought-resistant plant, comprising:

(a) constructing a recombinant vector in which a nucleotide sequence coding for an ABA carrier composed of two repeats of six transmembrane domains and one ATP-binding domain, and a nucleotide sequence having a homology therewith is operably linked to a promoter; and

(b) introducing the recombinant vector into a plant cell or a plant tissue (see Examples 6 and 7 and FIGS. 2 to 5).

In the above methods for producing the transgenic plant, the recombinant vector-constructing step (a) may be carried out by constructing an expression cassette containing the nucleotide sequence operably linked to the promoter, and inserting the expression cassette into a suitable vector.

According to an embodiment, the nucleotide sequence encoding an ABC carrier composed of two repeats of six transmembrane domains and one ATP-binding domain, and the nucleotide sequence having a homology therewith may be manipulated to be overexpressed or down-regulated in the expression vector and the transgenic plant.

As used herein, the term “ABC (ATP-binding cassette) carrier” refers to a protein that utilizes the energy of ATP hydrolysis to perform the transportation of various substrates across membranes such as the uptake of nutrients into cells and the exportation of, for example, toxic materials out of cells.

The term “homology”, as used herein, refers to the similarity between nucleic acid (DNA) sequences or between amino acid sequences.

The term “RNA interference (RNAi)”, as used herein, is intended to refer to the suppression of the expression of a gene of interest by DNA constructs which are homologous to a part of the DNA and are designed to produce short hairpins interfering with the RNAs.

RNA interference (RNAi) has an important role in defending cells against viruses and results in the cleavage of viral dsRNA. The mechanism is as follows: 1) Dicer cleaves hairpin dsRNA molecules into small-interfering RNA (siRNA) with 21˜23 nucleotide; 2) Dicer aids to integrate the siRNA into the RNA-induced silencing complex (RISC); 3) The siRNA-integrated RISC recognizes antisense mRNA complementary to the siRNA and induces the cleavage of the mRNA. Thus, antisense mRNA complementary to the siRNA is degraded.

This mechanism is applied in this invention. For example, a DNA construct which is designed on the basis a partial sequence of a target gene, e. g. AtPDR12 (SEQ ID NO: 1) to produce dsRNA, is inserted into a vector (refer to Example 4) which is then transformed into a plant to give an RNAi transgenic plant in which AtPDR12, and its highly homologous sequences AtPDR4 and AtPDR10 are down-regulated.

The partial sequence of AtPDR12 gene (SEQ ID NO: 1) may be the following sequence:

(SEQ ID NO: 13)
GCAAATCCTTCCATCATATTCATGGATGAACCTACTTCAGGATTGGA
TGCACGAGCTGCTGCCATCGTTATGAGGACTGTAAGGAACACAGTTGACA
CTGGTAGAACAGTCGTCTGCACCATTCACCAGCCTAGCATCGACATCTTT
GAAGCCTTTGATGAGTTGTTCCTACTTAAGCGTGGAGGTGAGGAGATATA
CGTTGGACCTCTTGGCCACGAATCAACCCATTTGATCAACTATTTTGAGA
GTATTCAAGGAATCAACAAGATCACAGAAGGATACAACCCAGCAACCTGG
ATGCTTGA

In other words, RNAi is a gene silencing process in which siRNA recognizes and binds to a specific sequence of mRNA and the target mRNA is degraded. Also, the siRNA binds to mRNAs which have sequences not only identical, but also similar (in this case, other genes with homology to AtPDR12) to those of the siRNA, and thus induces cleavage of the mRNA.

As used herein, the term “transgenic plant” is a plant which has foreign DNA sequence therein and whose DNA is modified using genetic engineering techniques in such a manner as to express, overexpress, or down-regulate the foreign DNA sequence.

As used herein, the term “ABA” refers to a plant hormone that is a weak acid containing 15 carbons and that regulates the growth and development of plants, such as embryo maturation, seed dormancy and germination, lateral root formation, cell division and extension and is involved in the responses to environmental stress factors such as drought, salinity, coldness, infection by pathogens, and UV light (reviewed in Leung and Giraudat, 1998; Rock, 2000).

The term “ABA carrier”, as used herein, refers to a transmembrane protein spanning the lipid-bilayer membrane that is involved directly or indirectly in the uptake and exclusion of the plant hormone ABA.

The term “salt- and/or drought-resistant protein”, as used herein, refers to a protein that mediates a process so as not to inhibit the growth of plants in the presence of a high concentration of salts or in arid regions. The term “salt” means all compounds in which the anion of an acid is combined with the cation of a base, as well as sodium chloride (NaCl), present in culture environments such as soil, water and air. Examples of the salt include:

Sodium salts: NaNO₃, NaCl, Na₂S, Na₂SO₄, Na₂CO₃, etc.

Potassium salts: KNO₃, KCl, K₂S, K₂SO₄, K₂CO₃, etc.

Ammonium salts: NH₄NO₃, NH₄Cl, (NH₄)₂S, (NH₄)₂SO₄, (NH₄)₂CO₃, etc.

Magnesium salts: Mg(NO₃)₂, MgCl₂, MgS, MgSO₄, MgCO₃, etc.

Barium salts: Ba(NO₃)₂, BaCl₂, BaS, BaSO₄, BaCO₃, etc.

Calcium salts: Ca(NO₃)₂, CaCl₂, CaS, CaSO₄, CaCO₃, etc.

Others: Pb(NO₃)₂, PbCl₂, PbS, PbSO₄, PbCO₃, AgNO₃, AgCl, Ag₂S, Ag₂SO_{4, Ag}₂CO₃, etc.

Each of the ABA carrier, the composition for the transportation of ABA and the composition for improving plants in resistance to salinity and drought, comprises an ABC carrier consisting of two repeats of six transmembrane domains and one ATP-binding domain or a nucleotide sequence coding for the ABC carrier.

In a preferred embodiment, the ABC carrier is derived from Arabidopsis thaliana and may be an AtPDR12 protein having an amino acid sequence of SEQ ID NO: 2 and may be a protein whose amino acid sequence shares a homology of at least 60%, e.g., 70%, preferably at least 80%, more preferably 90% to 95%, and most preferably 95% to 99% with that of SEQ ID NO: 2. For example, the homologue of AtPDR12 may be selected from the group consisting of AtPDR3 of SEQ ID NO: 4, AtPDR4 of SEQ ID NO: 6, AtPDR6 of SEQ ID NO: 8, AtPDR10 of SEQ ID NO: 10, and AtPDR13 of SEQ ID NO: 12.

The nucleotide sequence encoding the ABC carrier may be a sequence coding for the AtPDR12 of SEQ ID NO: 2 or for a protein whose amino acid sequence shares a homology of at least 60%, e.g., 70%, preferably at least 80%, more preferably 90% to 95%, and most preferably 95% to 99% with that of SEQ ID NO: 2. For example, the nucleotide sequence encoding the ABC carrier is a nucleotide sequence of SEQ ID NO: 1 or a nucleotide sequence sharing a homology of at least 60%, e.g., 70%, preferably at least 80%, more preferably 90% to 95%, and most preferably 95% to 99% with that of SEQ ID NO: 1. In one embodiment, the homologous nucleotide sequence may be selected from the group consisting of nucleotide sequences coding for the AtPD3 protein having an amino acid sequence of SEQ ID NO: (e.g. SEQ ID NO: 3), for the AtPD4 protein having an amino acid sequence of SEQ ID NO: 6 (e.g. SEQ ID NO: 5), for the AtPD6 protein having an amino acid sequence of SEQ ID NO: 8 (e.g. SEQ ID NO: 7), for the AtPD10 protein having an amino acid sequence of SEQ ID NO: 10 (e.g. SEQ ID NO: 9), and for the AtPD13 protein having an amino acid sequence of SEQ ID NO: 12 (e.g. SEQ ID NO: 11).

The homologous sequences are summarized in the following table.

| | Homology to | | | |
|---|---|---|---|---|
| | AtPDR12 | | Accession | |
| | (gene/ | AGI No. | No. | SEQ ID NO: |
| | protein) | (gene) | (protein) | (protein/gene) |
| AtPDR3 | 60%/62% | At2g29940 | NP_180555 | SEQ ID NO: 3/4 |
| AtPDR4 | 61%/62% | At2g26910 | NP_180259 | SEQ ID NO: 5/6 |
| AtPDR6 | 62%/66% | At2g36380 | NP_181179 | SEQ ID NO: 7/8 |
| AtPDR10 | 61%/60% | At3g30842 | NP_683617 | SEQ ID NO: |
| | | | | 9/10 |
| AtPDR13 | 61%/70% | At1g15215 | NP_680694 | SEQ ID NO: |
| | | | | 11/12 |

In accordance with still yet another aspect thereof, the present invention provides an expression cassette in which an ABC carrier gene or a nucleotide sequence sharing homology therewith is operably linked to a promoter, and a recombinant vector carrying the same.

In an embodiment, the expression cassette or the recombinant vector may comprises a promoter, a gene encoding the ABC carrier, e.g., a gene encoding AtPDR12, or a homologue thereof, and a transcription terminator. Any promoter which is expressed in a plant may be used. For example, the promoter may be selected from the group consisting of an AtPDR12 promoter, a CMV (Cauliflower Mosaic Virus) 35S promoter, an nos (nopaline synthase) promoter of Agrobacterium tumefaciens Ti plasmid, an ocs (octopine synthase) promoter, an mas (mannopine synthase) promoter, a ubiquitin promoter, an actin promoter, a rubisco promoter, an RD29A promoter, and any other known plant expression promoter. Preferable for overexpression is a CMV 35s promoter, a ubiquitin promoter, an actin promoter or a rubisco promoter. A stress-inducible promoter, (e.g. RD29A promoter), may be also used. However, the present invention is not limited to the examples. So long as it allows overexpression in plant cells, any promoter or enhancer may be used. For example, a 35s promoter enhancer for enhancing transcription, or an AtADH 5′ UTR (sequence in the 5′-untranslated region of the Arabidopsis ADH gene) enhancer, or an OsADH 5′-UTR (sequence in the 5′-untranslated region of the rice ADH gene) enhancer for promoting translation may be used. As for the transcription terminator, it may be any of those which are typically used in plant cells. An illustrative, non-limiting example is a nopaline synthase (nos) transcription terminator.

The expression cassette or recombinant vector of the present invention may further comprise a marker which is indicative of the expression of an AtPDR12 gene or allows the selection of a transgenic plant expressing the gene. Examples of the marker gene useful in the present invention include, but are not limited to, genes resistant to antibiotics such as kanamycin, spectinomycin, basta, hygromycin, gentamicin and bleomycin, or genes coding for GUS (β-glucuronidase), CAT (chloramphenicol acetyltransferase), luciferase and GFP (green fluorescent protein).

When introduced along with the expression cassette into plants, the marker allows the selection of transgenic plants appearing during the growth in a medium containing a specific antibiotic or showing a blue color (GUS), luminescence (luciferase), as a result of reaction with a substrate or fluorescene.

The recombinant vector may be constructed by inserting the expression cassette into the backbone of a known vector and comprises a sequence encoding an ABC carrier able to transport ABA. In the recombinant vector, the sequence is operably linked to transcription and translation factors and designed to be expressed in plants, plant tissues or plant cells and optionally overexpressed or down-regulated. In an embodiment, the recombinant vector may be designed to allow the ABC carrier to be expressed, overexpressed or down-regulated.

Examples of the known vector include pBI121, pHellsgate8, pROKII, pBI76, pET21, pSK(+), pLSAGPT, pUC, and pGEM. In addition, the vector expressed in plants comprising a CMV35s promoter such as pCAMBIA series (pCAMBIA1200, 1201, 1281, 1291, 1300, 1301, 1302, 1303, 1304, 1380, 1381, 2200, 2201, 2300, 2301, 3200, 3201, 3300), pMDC32, and pC-TAPa-pYL436, may be used.

In an embodiment, the transgenic plant may be designed to allow the ABC carrier to be expressed, overexpressed or down-regulated. For example, the overexpression of the ABC carrier may be achieved by a recombinant vector constructed to use a strong promoter such as a CMV 35S promoter, an actin promoter, a ubiquitin promoter, a rubisco promoter, or an RD29A promoter, or an enhancer such as a 35s promoter enhancer for enhancing transcription, ATADH 5′UTR (a sequence in the 5′-untranslated region of the Arabidopsis thaliana ADH gene) enhancer, or an OsADH 5′-UTR (a sequence in the 5′-untranslated region of the rice ADH gene) enhancer for promoting translation. The suppression of expression may be carried out by RNAi, that is, by adding siRNA or antisense RNA complimentary to a partial or full sequence of the ABC carrier gene, inserting T-DNA or endogenous transposon, or mutating the gene at a transcription level or a translation level through modification or deletion of a partial or full nucleotide or one amino acid sequence of the gene to induce the loss of the function.

Also, the present invention provides a transgenic plant which is transformed with the recombinant vector. The transgenic plant comprises an ABC carrier-encoding sequence which is designed to be operably linked to and controlled by transcription and translation factors. The transgenic plant is of plants, preferably a plant, a plant cell or a plant tissue. The plant tissue includes a plant seed. The plant may be a herbaceous or woody plant, examples of which include, but are not limited to, flowering plants, garden plants, onion, carrot, cucumber, olive, sweet potato, potato, Chinese cabbage, radish, lettuce, broccoli, tobacco, petunia, sunflower, Brassica napus, leaf-mustard, Arabidopsis thaliana, Brassica campestris, Brassica juncea, Nicotiana tabacum, Betula platyphylla, poplar, cross-bred poplar (e.g., Populus alba X P. tremula var. glandulosa: a natural crossbred Populus alba and P. tremula var. glandulosa), and Betula schmidtii. The plant may be asexually propagated using a method selected from the group consisting of somatic embryogenesis, tissue culture and cell line culture.

The transgenic plant may be prepared using any well-known technique. A typical, non-limiting example is Agrobacterium tumefaciens-mediated DNA transfer. More preferably, recombinant agrobacterium prepared by electroporation, microparticle injection or gene gun is induced to infect a host plant cell by soaking method.

As described, the ABC carriers according to the present invention may transport ABA or inhibit the transportation of ABA. The control of their expression may result in an improvement in resistance to salinity and/or drought.

For example, the overexpression of at least one of the ABC carrier group consisting of AtPDR12 (SEQ ID NO: 2), AtPDR3 (SEQ ID NO: 4), AtPDR4 (SEQ ID NO: 6), AtPDR10 (SEQ ID NO: 10), and homologues thereof with a homology of at least 60%, preferably at least 80%, more preferably at least 90% to 95%, and most preferably at least 95% to 99% may allow the preparation of a transgenic plant which is improved in resistance to stress including salinity and/or drought and in ability to transport ABA. A transgenic plant in which at least one gene selected from the group consisting of an AtPDR12 gene (SEQ ID NO: 1), an AtPDR3 gene (SEQ ID NO: 3), an AtPDR4 gene (SEQ ID NO: 5) and an AtPDR10 gene (SEQ ID NO: 9) is overexpressed exhibits an improvement in resistance to stress including salinity and drought and in ability to transport ABA. The overexpression may be performed as mentioned above.

A transgenic plant in which neucleotide sequences coding AtPDR6 (SEQ ID NO: 8), AtPDR13 (SEQ ID NO: 12), and homologues thereof with a homology of at least 60%, preferably at least 80%, and more preferable at least 90% to 99% are down-regulated or inactivated closes its stomatal pores rapidly under arid conditions, showing improved drought tolerance. For example, the down-regulation of the AtPDR6 gene (SEQ ID NO: 7) and/or the AtPDR13 gene (SEQ ID NO: 11) may allow the preparation of a transgenic plant which has improved resistance to salinity and drought and improved ability to transport ABA.

Therefore, the transgenic plant is transformed by a modified form of the target gene or regulated the expression level of the target genes. Target genes mean the genes which regulate stomata movement through the change of ABA transportation activity. The transgenic plant can either readily translocate pollutants such as heavy metals from roots to shoots along with water upon activated transpiration or can have improved drought tolerance.

Therefore, in accordance with another aspect thereof, the present invention provides a method for environmental remediation in highly salty and/or arid regions, comprising planting the transgenic plant in the soil. In accordance with another aspect thereof, the present invention provides a method for increasing crop productivity in highly salty and/or arid regions, comprising planting the transgenic plant.

### DESCRIPTION OF DRAWINGS

FIG. 1 shows the tissue-specific expression of AtPDR12 in the Arabidopsis thaliana plant transformed with AtPDR12 promoter: uidA as analyzed by GUS labels. The GUS activity driven AtPDR12 promoter is observed in both roots and leaves, particularly strongly in lateral roots and guard cells (A; 2-week-old Arabidopsis seedling, B; mature leaves, C; guard cells, D; flowers, E; anther and pollen, F; an embryo germinated from a seed, G; root tissue, H; silique, I; seeds within siliques, J; an end of a main root, K; developing lateral root, L; lateral root.)

FIG. 2 shows AtPDR12 contributes to increase of the salt tolerance of the plant. (A) is the expression level of AtPDR12 gene in the AtPDR12-overexpressing Arabidopsis by RT-PCR. (B) to (D) showed the growth extent (B), fresh weight (C) and root length (D) of the AtPDR12-overexpressing Arabidopsis thaliana was cultured in media containing 120, 130 and 140 mM NaCl, respectively.

FIG. 3 is a graph of germination rates when seeds of the AtPDR12-overexpressing Arabidopsis thaliana transgenic plant were induced to germinate in a medium containing 200 mM NaCl, showing an improved resistance of the plant to salinity.

FIG. 4 is of photographs of leaves of the AtPDR12-knockout mutant plant(atpdr12) after they were cultured in arid regions, showing the involvement of AtPDR12 in drought tolerance.

FIG. 5 shows the deletion of AtPDR12 gene induced decrease in sensitivity to ABA in graphs where the inhibition of ABA against light-induced opening of stomata is measured (A), the ABA-induced stomata closure is measured (B), germination rates are measured upon the induction of germination in an ABA-containing medium (C) and the number of lateral roots formed in an ABA-containing medium is counted (D).

FIG. 6 is a graph in which the time dependent content of radiolabeled ABA taken up into protoplasts isolated from the AtPDR12-knockout mutant plant(atpdr12-1) and the wild-type are incubated in a betain solution containing radiolabeled ABA, showing that AtPDR12 mediates the uptake of ABA into the plant cell.

FIG. 7 shows the gene expression of the plants down-regulated in the expression of the AtPDR12 gene and its homologues AtPDR4 and AtPDR10 genes (RNAi), in which the homology among the three genes is given (A) (AtPDR4: 61% homology, AtPDR10: 61% homology) and the expression levels of the three genes in the down-regulated plant are measured by RT-PCR.

FIG. 8 is graphs of the germination rates of AtPDR3/AtPDR4-knockout mutant plant (pdr3-1, pdr3-2, and pdr4), both having a homology of 62% with the AtPDR12 amino acid sequence, upon the induction of germination in an ABA-containing medium, showing that the mutant plant (pdr3-1, pdr3-2 and pdr4) decrease in sensitivity to the ABA.

FIG. 9 is of graphs of the germination rates of AtPDR6-or AtPDR13-knockout mutant plant(pdr6-1, pdr6-2, and pdr13), both having respective homologies of 65 and 70% with the AtPDR12 nucleotide sequence, upon the induction of germination in an ABA-containing plant, showing that the mutant plants(pdr6-1, pdr6-2, and pdr13) increase in sensitivity to the ABA.

FIG. 10 is of graphs in which the number of siliques and the weight of seeds are measured in mutant plants when they are cultured for 14 days without being watered after completion of nutritional growth (5 weeks), showing that the AtPDR12-overexpressing transgenic Arabisopsis thaliana is resistant to drought in terms of seed productivity.

FIG. 11 is of graphs in which stomata of the AtPDR3-knockout mutant plant exhibit hypersensitive movement in response to ABA compared to that of the wild-type (A) whereas stomata of the AtPDR2- or AtPDR4-knockout mutant plant is hyposensitive to ABA, compared to that of the wild-type (B), showing that sensitivity to ABA is changed by the knockout of PDR genes homologous to AtPDR12.

### BEST MODE

In the following, the present invention is described in detail through experiments. The experiments are not intended to limit the technical spirit of the present invention, but are intended to describe the invention

### EXAMPLE 1

### Plant Culture Conditions

Seeds of wild type of Arabidopsis thaliana (dry seeds within one year after harvest), and seeds of the transgenic Arabidopsis thaliana species, species prepared in the following Examples 3 and 4, which overexpressed(e.g., AtPDR12), or down-regulated a certain gene, or seeds of knockout mutant Arabidopsis thaliana species (atpdr12-1 (SALK_{—}013945), atpdr12-2 (SALK_{—}005635), purchased from SALK, dry seeds within one year after harvest) were surface sterilized with 70% (v/v) ethanol (Samchun) and 25% (v/v) Chlororox (Yuhan Clorox) and stored at 4° C. for two days in a dark place before sawing on 1/2 MS-agar medium (Murashige and Skoog Duchefa). And then the media culture on a vertically or horizontally for two to three weeks (culture condition (day/night): 22/18° C., 16/8 h, light condition: 40 μmol m⁻² s⁻¹).

### EXAMPLE 2

### Determination of Gene Expression Tissue Using Promoter-GUS Fusion Protein

PCR was performed on a genomic DNA extracted from wild-type Arabidopsis thaliana, in the presence of a pair of primers containing a HindIII restriction enzyme site (5′-AAGCTTACGCCGGCCGCCGCCGCGGCAG-3′, SEQ ID NO: 14) and a BamHI restriction enzyme site (5′-GGATCCTTTGTATCCAAGAAATCAAAGT-3′, SEQ ID NO. 15), respectively, to amplify an AtPDR12 promoter which was then inserted into a pBII01.2 vector (Clontech) using the restriction enzymes HindIII (Roche) and BamHI (Roche) to construct a recombinant vector for transformation. This was introduced into Agrobacterium using electroporation. The transformed Agrobacterium thus obtained was transformed into Arabidopsis thaliana using the floral dipping method (Clough and Bent, 1988). The seeds of transgenic Arabidopsis thaliana were selected on a 1/2 MS-agar medium containing 30 μg/L of kanamycin (Duchefa), and the seeds were harvested from the plants that survived. T2 Seeds from the T1 generation Arabidopsis thaliana were used for GUS analysis.

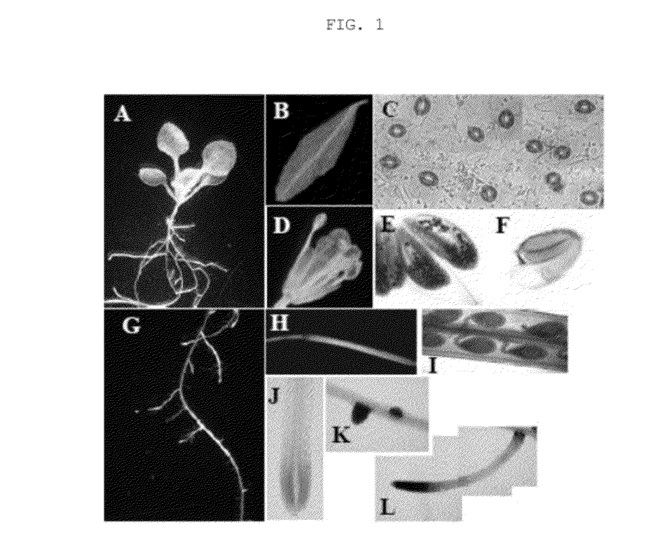

The transgenic Arabidopsis thaliana grown for two weeks on the 1/2 MS-agar medium was incubated for 24 hours in 100 mM phosphate buffer containing 0.5 mM K4Fe(CN)6 (Sigma), 0.5 mM K3Fe(CN)6 (Sigma), 10 mM EDTA (USB), 0.1% (v/v) Triton X-100 (Sigma) and 500 mg/mL X-Gluc. (Duchefa), followed by removal of chlorophyll with 100% (v/v) ethanol. The plant was observed using an optical microscope and the images are shown in FIG. 1. As shown in FIG. 1, the AtPDR12 promoter-GUS was observed to be expressed throughout the plant, such as in the leaves, flowers, lateral roots, seeds, and guard cells.

### EXAMPLE 3

### Generation of Transgenic Arabidopsis Thaliana

cDNA was synthesized from total RNA extracted from wild-type Arabidopsis thaliana using a reverse transcriptase (Clontech) (see Example 5, below). With the cDNA serving as a template, PCR was performed in the presence of an AtPDR12-TFF primer (5′-CCCGGGGGGGATCCATGGAGGGAACTAGITTICACCAAGCGAGTA-3′, SEQ ID NO: 16) and an AtPDR12-TFR primer (5′-GGATCCGCGGCCGCCIATCGTITTIGGAAATTGAAACTCTIGATTC-3′, SEQ ID NO: 17) to amplify AtPDR12 DNA (SEQ ID NO: 1) which was then inserted into a T-vector (Promega). Again, the AtPCR12 DNA was cloned into a pBI121 vector (Clontech) to construct a recombinant vector Using BamHI restriction enzyme. This pBI121 vector was used as a plant expression vector for use in the generation of transgenic Arabidopsis thaliana and has a CaMV35S Promoter, multiple cloning sites and a nopaline synthetase terminator.

The recombinant vector was introduced into Agrobacterium by electroporation with which Arabidopsis thaliana was then transformed using the floral dipping method (Clough and Bent, 1988). The Seeds of transgenic Arabidopsis thaliana was selected on a 1/2 MS-agar medium containing 30 μg/L of kanamycin (Duchefa), and seeds were harvested from the plants that survived.

Seeds of the T3 generation homozygote were used for phenotype analysis.

### EXAMPLE 4

### Generation of RNAi Transgenic Plant

To generate an RNAi (RNA interference) transgenic plant in which the expression of AtPDR12 and its homologues AtPDR4 (SEQ ID NO: 5) and AtPDR10 (SEQ ID NO: 9) was suppressed, first, PCR was performed in the presence of an Ri-F primer (5′-GGGGACAAGITIGTACAAAAAAGCAGGCTICATGGCAAACCCTICTATAGTATICATGGATG-3′) and an Ri-R primer (5′-GGGGACCACTTIGTACAAGAAAGCTGGGICITAATCAAGCATCCATGCTGCCGGATTATTG-3′). The PCR product thus obtained was inserted into a pDONR221 vector (Invitrogen) using gateway BP clonase and then cloned into the binary vector pHellsgate8 (Invitrogen) using gateway LR clonase (Invitrogen) to give a recombinant vector (pHellsgate8-AtPDR12Ri). The pHellsgate8-AtPDR12Ri vector was transformed into Arabidopsis thaliana through the mediation of Agrobacterium to afford an AtPDR12 RNAi-transgenic plant according to example 3. The Seeds of transgenic Arabidopsis thaliana was selected on a 1/2 MS-medium containing 30 μg/L kanamycin (Duchefa) and seeds were harvested from the plants that survived.

### EXAMPLE 5

### RNA Isolation and RT-PCR

Total RNA was extracted from two- to three-week-old wild-type Arabidopsis thaliana using Trizol. In detail, the plant was cultured on a 1/2 MS-agar medium (culture condition (day/night): 22/18° C., 16/8 h, light condition: 40 μmol m⁻² s⁻¹), and evenly ground using liquid nitrogen, followed by isolating total RNA with the Trizol (Phenol in saturated buffer (pH 4.3, USB), guanidine thiocyanate (Sigma), Ammonium thiocyanate (Sigma), sodium acetate (3 M, pH 5.0, Aldrich), Glycerol (USB), and 8-hydroxyquinoline (Sigma)). It was used for RT-PCR as template.

cDNA was synthesized from 5 μg of the RNA (using a Powerscript RT (reverse transcription) kit (Clontech) and an oligo-dT primer 5′-TTTTTTTTTTTTTTTTTT-3′, SEQ ID NO: 20). With 2 μL of the cDNA serving as a template, PCR was preformed in the presence of primers specific for AtPDR4, AtPDR10, and AtPDR12, respectively. β-tubulin and ubiquitine were used as loading controls.

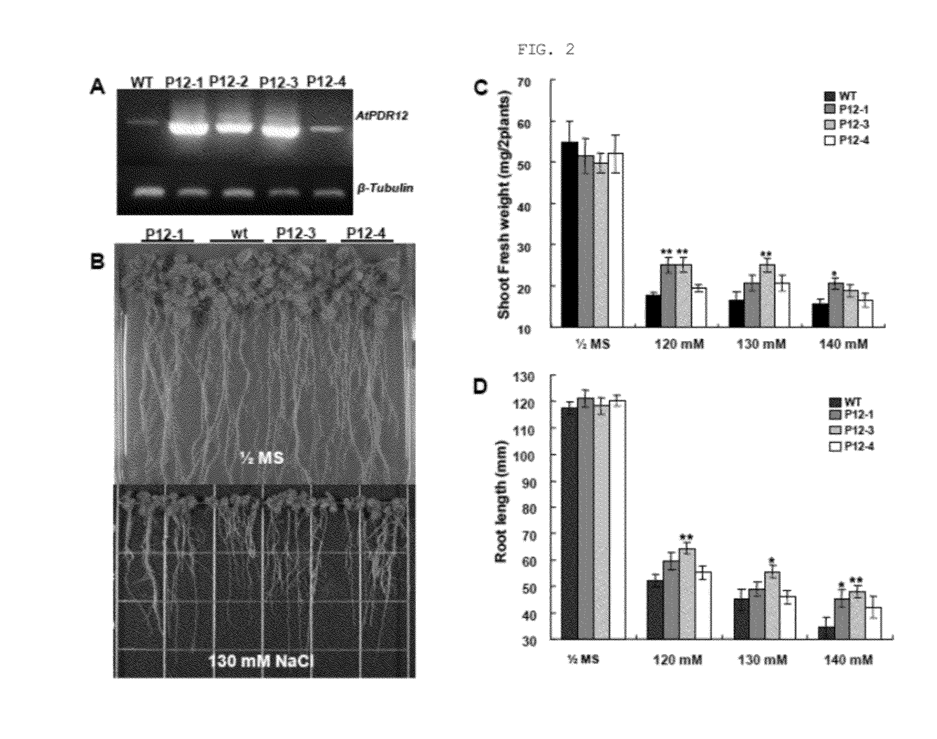

In greater detail, to examine the expression of the AtPDR12 gene in the AtPDR12-transformed plant, cDNA was synthesized from each of wild-type, and the overexpressed transgenic plants prepared in Example 3 (four plants were randomly selected and named P12-1, P12-2, P12-3 and P12-4) (see Example 5) and used as a template for PCR using a pair of primers P123rd-F (5′-CTGCTTTTGGGTCCTCCAAGTTCT-3′, SEQ ID NO: 21) and P123rd-R (5′-GAGATTGAATGTCTCTGGCGCAG-3′, SEQ ID NO: 22). The PCR result is given in FIG. 2A. As shown in FIG. 2A, a high expression level of AtPDR12 was detected in the AtPDR12 transgenic plant. As a loading control, β-tubulin was not significantly different in expression level between the wild-type and the transgenic plants (primers: Tub-F (5′-GCTGACGTTTTCTGTATTCC-3′, SEQ ID NO: 23), Tub-R (5′-AGGCTCTGTATTGCTGTGAT-3′, SEQ ID NO: 24).

The expression level of AtPDR12 and its homologues AtPDR4 and AtPDR10 in the AtPDR12 RNAi plant prepared in Example 4 was examined. In this regard, first, cDNA was synthesized from the wild-type plant and the transgenic plants.

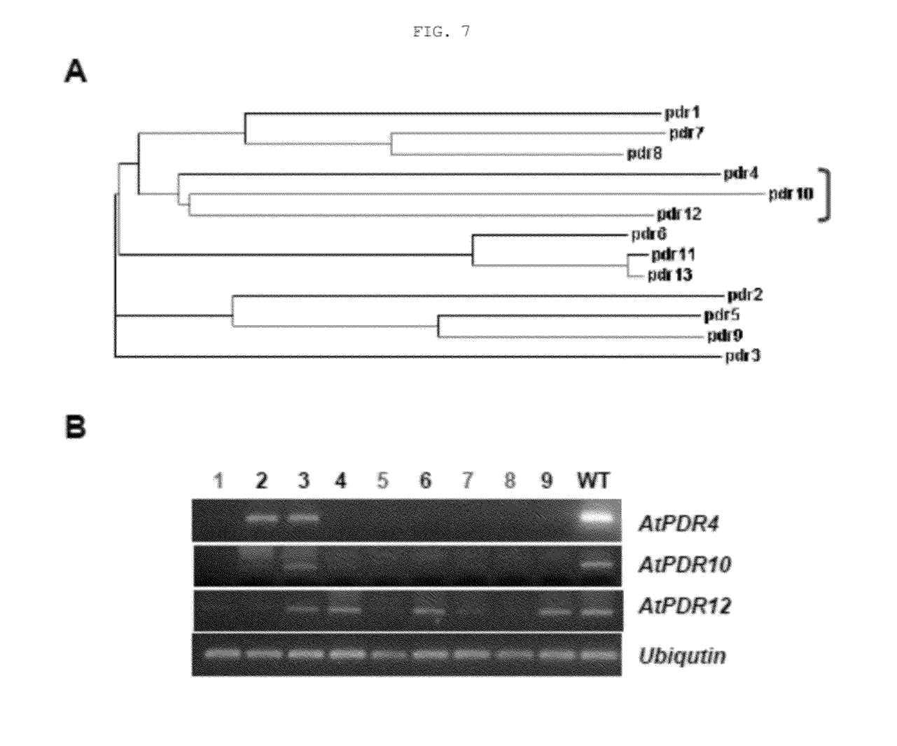

(1-9: AtPDR12-down-regulated plants (AtPDR12 RNAi) surviving 30 μg/L kanamycin (Duchefa) on a 1/2 MS-agar medium was randomly selected and numbered 1 to 9) and used as templates for PCR using respective primer sets specific for AtPDR4 (F-GCATTAGTGGGAGTAAGTGGTGCC (SEQ ID NO: 25), R-TTGAGTGTCCCTTTTGGAGCCAA (SEQ ID NO: 26)), AtPDR10 (F-GAATGGATTAAGCGGTGCTTTTAG (SEQ ID NO: 27), R-TTGACATCGCGCCTACACTATTGA (SEQ ID NO: 28)), and AtPDR12 (F-CTGCTTTTGGGTCCTCCAAGTTCT (SEQ ID NO: 29), R-GAGATTGAATGICTCTGGCGCAG-3′ (SEQ ID NO: 30)). PCR results are shown in FIG. 7B. As shown in FIG. 7B, the AtPDR12-down-regulated plants (1, 5, 7, 8) were lower in the expression level of the gene than was the wild-type. As a loading control, ubiquitin was amplified (primers: LP-GCCAAGATCCAAGACAAAGA (SEQ ID NO: 31), RP-TTACGAGCAAGCATCATCAA (SEQ ID NO: 32)) and there were no significance differences in the expression level of ubiquitin between the wild-type and the transgenic plants.

### EXAMPLE 6

### Assay for Resistance to Salinity and Drought

The AtPDR12 transgenic plants were assayed for resistance to salinity and drought. In this regard, first, the wild-type and the overexpressed plants prepared in Example 3 were cultured for three weeks on 1/2 MS-medium containing 120 mM, 130 mM and 140 mM NaCl, respectively (culture condition (day/night): 22/18° C., 16/8 h, light condition: 40 μmol m⁻² s⁻¹). In FIG. 2, the culture results are shown for growth extent (B), fresh weight (C) and root length (D). As is apparent from the data of FIG. 2, the overexpressed plants were superior to the wild-type plant in terms of growth extent, fresh weight and root length and thus more resistant to salinity than the wild-type plant.

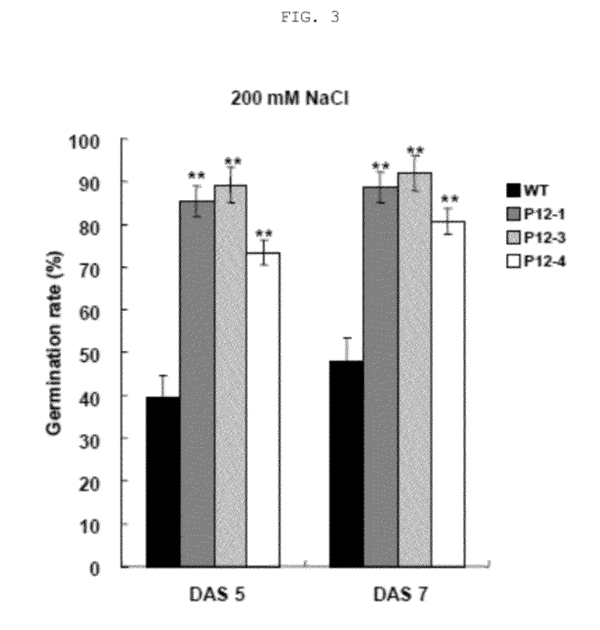

In addition, seeds of the wild-type and the AtPDR12-overexpressed plants prepared in Example 3 were induced to germinate on a 1/2 MS-agar medium containing 200 mM NaCl. The results are presented in FIG. 3. As shown in FIG. 3, the higher germination rates detected in the overexpressed plants demonstrated that the overexpressed plants became more resistant to salinity than the corresponding wild-type.

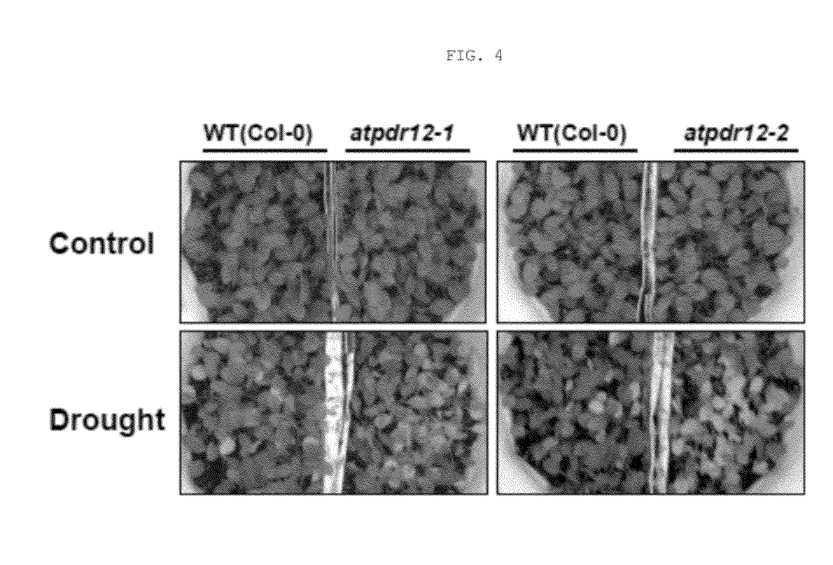

As for assay for resistance to drought stress, its results are shown in FIG. 4 after an AtPDR12-knockout mutant plant (atpdr12), a plant which does not express AtPDR12 due to the insertion of “T-DNA” into the AtPDR12 gene sequence, which was purchased from SALK. Mutants (plants different in the sites at which T-DNA was inserted into the AtPDR12 gene sequence were randomly named atpdr12-1 and atpdr12-2. Respective SALK numbers are as follows: atpdr12-1 (SALK_{—}013945) and atpdr12-2 (SALK_{—}005635)) and the wild-type were grown for four weeks in soli and additionally for two weeks withdraw watering. A control was cultured and watered every two or three days. As shown in FIG. 4, leaves of the AtPDR12-knockout mutant plant (atpdr12) wilted faster than those of the wild-type. Hence, the AtPDR12-knockout mutant plant became weak in drought tolerance and was more sensitive to drought stress, compared to the wild-type. From this data, it is expected that AtPDR12-overexpressed transgenic plants shows a higher resistance to drought than does the wild-type.

### EXAMPLE 7

### Assay for Response to ABA, an Indicator of Drought Tolerance

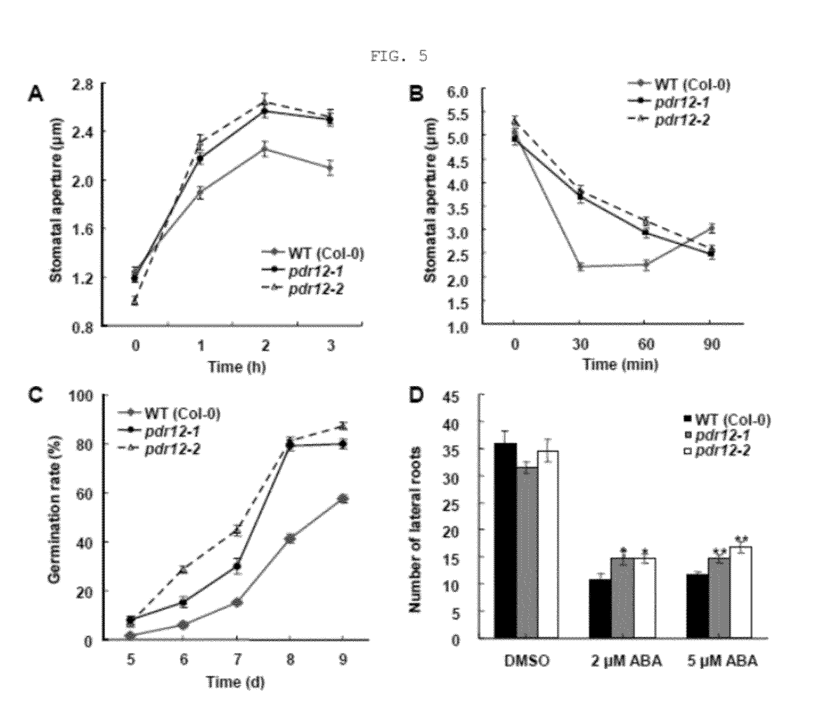

Leaves of five-week-old Arabidopsis thaliana were floated on an ABA-containing buffer (1 μM ABA (Sigma), 10 mM MES (Duchefa), 10 mM KCl (Sigma) pH 6.05 buffer). After exposure to light (170 μmol m⁻² sec⁻¹ white light at 23° C.) for predetermined times, epidermal strips were peeled from leaves and observed on slide glass using a microscope to measure the degree of stomatal opening. The results are shown in FIG. 5A. Leaves that had been floated on a buffer in the absence of ABA under light (170 μmol m⁻² sec⁻¹ white light at 23° C.) for 3 hours were transferred onto a 50 μM ABA-containing buffer. The stomatal closures of the leaves were measured with time dependency. The results are shown in FIG. 5B. Stomata opening movement by light of AtPDR12-knockout mutant plants (atpdr12) were not inhibited as much as the wild type (FIG. 5A). Furthermore, stomata of atpdr12 plants closed more slowly in response to ABA(FIG. 5B).

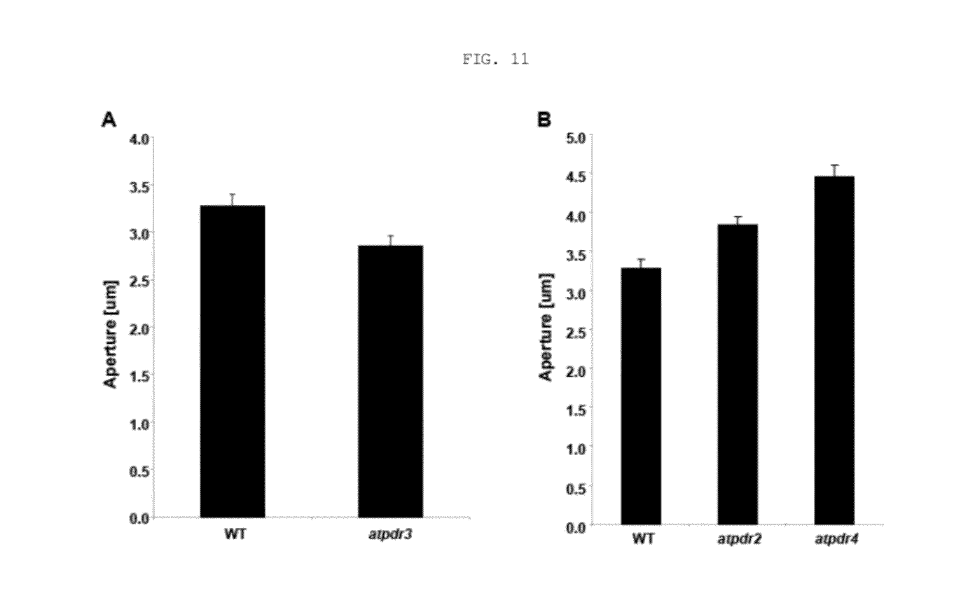

Under the same condition with the exception the ABC concentration as 3 μM, AtPDR12-knockout mutant plants and its homologues were monitored ABA induced stomata closing and the results are given in FIG. 11. Stomata of the AtPDR3-knockout mutant plant (purchased from SALK) exhibited hypersensitive movement in response to ABA compared to that of the wild-type (FIG. 11A) whereas stomata of the AtPDR2- or AtPDR4-knockout mutant plants (purchased from SALK) was hyposensitive to ABA, compared to that of the wild-type (FIG. 11B).

When seeds were induced to germinate in a 1/2 MS-agar medium in the presence of 1 μM ABA, which is inhibitory of seed germination, higher germination rates were observed in the AtPDR12-knockout mutant plant (atpdr12) than in the wild-type (FIG. 5C). In addition, seedlings were grown on 1/2 MS-agar medium for four days in the absence of ABA and then transferred to the same medium supplemented with ABA (2 μM or 5 μM). 7 days after the transfer of the plants, 5˜8 more lateral roots were observed in atpdr12 than in the wild-type. These results indicate that atpdr12 is hyposensitive to ABA in comparison to the wild-type (FIG. 5D).

Therefore, tissues of the AtPDR12-knockout mutant plant(adtpdr12), such as seeds, lateral roots and guard cells, are understood to significantly decrease in response to ABA. That is, the overexpression or activation of the gene coding for AtPDR12 or its homologues makes the plant close stomata faster and to a greater extent and thus makes it more resistant to drought than the wild-type.

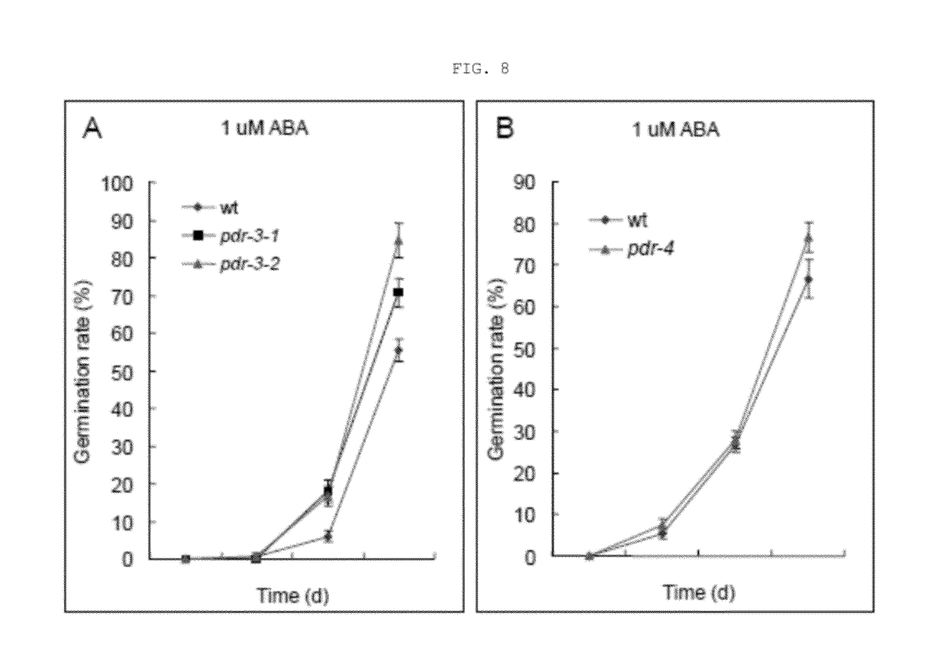

Further, after seeds were induced to germinate in a 1/2 MS-agar medium in the presence of 1 μM ABA, which is inhibitory of seed germination, the germination rates of the wild-type and the AtPDR3 (SEQ ID NO: 3)- or AtPDR4 (SEQ ID NO: 4)-knockout mutant plants(pdr3-1, pdr3-2, pdr-4, purchased from SALK) were measured and the results are shown in FIG. 8. As shown in FIG. 8, the mutant plants (pdr3-1, pdr3-2, pdr4) are higher in germination rate than is the wild-type.

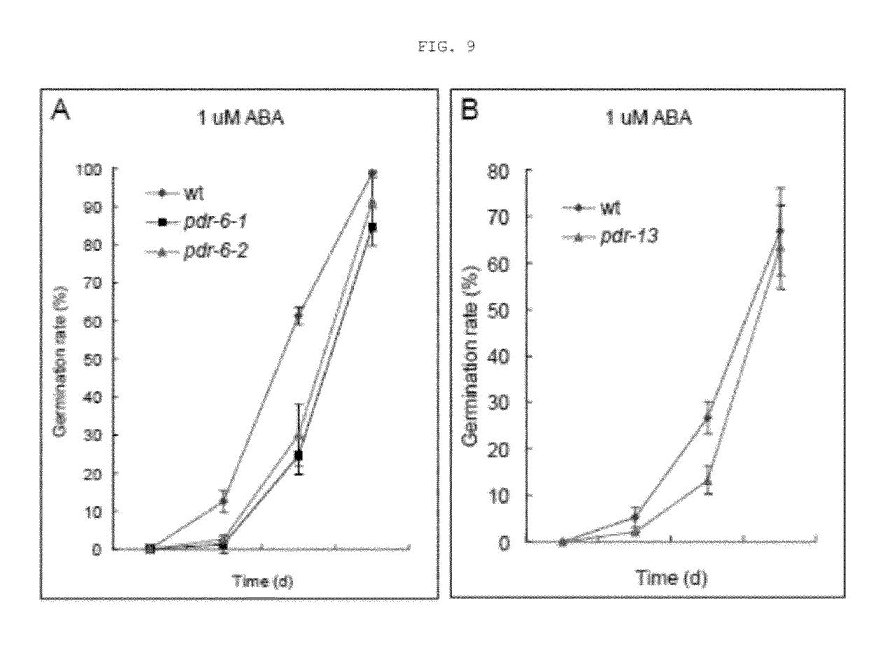

In the same medium as described above, seeds of the AtPDR6 (SEQ ID NO: 7)- or AtPDR13 (SEQ ID NO: 11)-knockout mutant plants (pdr6-1, pdr6-2, pdr13, purchased from SALK) lacking the expression of AtPDR6 (SEQ ID NO: 7) or AtPDR13 (SEQ ID NO: 11) were analyzed for germination rate and the results are showed in FIG. 9. As shown in FIG. 9, lower germination rates were observed in the AtPDR6- or AtPDR13-knockout mutant plant than in the wild-type.

In summary, seeds of the mutant plants were observed to have a decreased response to ABA if they lacked the expression of AtPDR3 or AtPDR4 (pdr3-1, pdr3-2, pdr-4) and to have an increased response to ABA if they lacked the expression of AtPDR6 or AtPDR13 (pdr6-21 pdr6-2, pdr13). As is apparent from the results, these genes are understood to be involved in ABA transport directly or indirectly. Therefore, the overexpression (AtPDR3, AtPDR4) or down-regulation (AtPDR6, AtPDR13) of the genes may lead to the development of the mutant plants which can close stomata faster and to a greater extent and thus are more resistant to drought than the wild-type.

### EXAMPLE 8

### ABA Transport Activity Assay

The AtPDR12 gene was assayed for ABA transport activity. In this regard, protoplasts of the wild-type and the AtPDR12-knockout mutant plant (adpdr12) were incubated for a predetermined period of time in a betaine solution (500 mM glycine betaine monohydrate (Sigma), 10 mM CaCl₂ (Sigma), 10 mM MES (Duchefa)) containing an isotope (³H)-labeled ABA (³H-ABA, Amersham Biosciences).

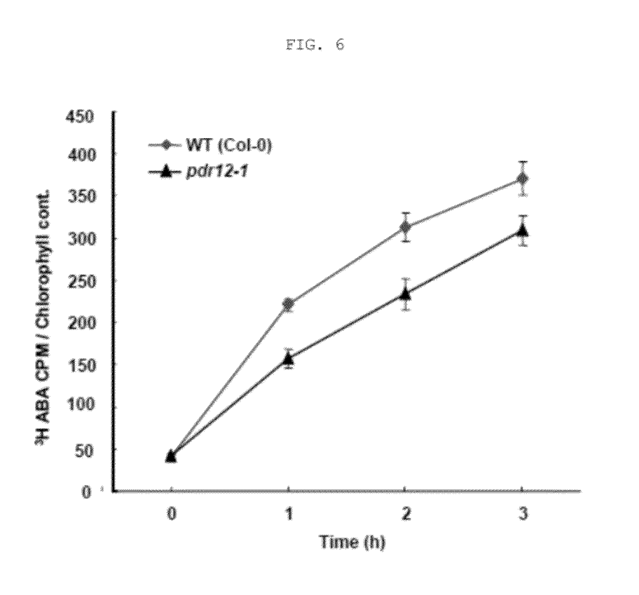

³H-ABA uptakes into the protoplasts were measured using a liquid scintillation counter (LS6500, Beckman) and the results are shown in FIG. 6. The protoplasts were harvested from the media containing the isotope-labeled ABA and analyzed for ABA content into the cells. As shown in FIG. 6, lower ABA levels had accumulated in the AtPDR12-knockout mutant plant(atpdr12) than in the wild-type, indicating that AtPDR12 mediates the uptake of ABA into cells.

Hence, the AtPDR12-overexpressing transgenic plants can rapidly take up ABA, which plays a crucial role in drought resistance, into the cells, and can be utilized to generate a plant which is improved in resistance to drought, salinity, cold, heat, infection by pathogens, etc.

### EXAMPLE 9

### Assay for Seed Productivity under Drought Stress

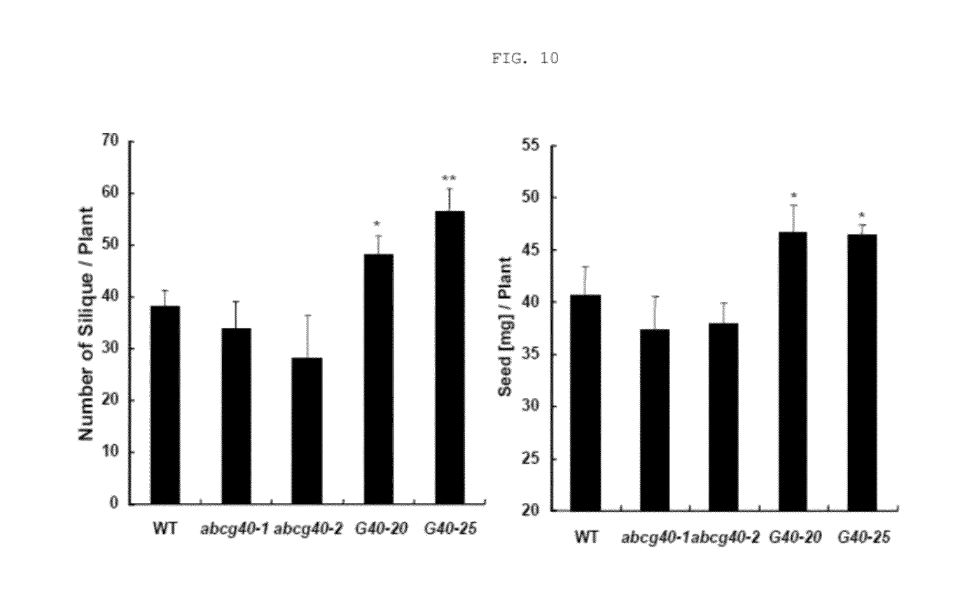

To assay seed productivity under drought stress, the AtPDR12-overexpressing transgenic Arabidopsis plants prepared in example 3 (randomly selected, named P12-20 and 25, respectively) and the AtPDR12-knockout mutant plant (atpdr12-1, 2) were cultured, together with the wild-type, in soil for 5 weeks and then for an additional 2 weeks without watering. The number of siliques(A) and the weight of seeds(B) in the plants were measured and the results are showed in FIG. 10. As is apparent from the data of FIG. 10, the AtPDR12-overexpressing transgenic Arabidopsis plants (P12-20, 25) showed higher seed productivity in terms of the number of siliques and the weight of seeds than did the AtPDR12-knockout mutant plant (atpdr12-1, 2) and the wild-type.

As described above, the AtPDR12 protein according to the present invention is involved in the transportation of the ABA hormone, which plays a crucial role in drought tolerance. The overexpression of AtPDR12 gene leads to the development of a novel plant which is improved in resistance to various stresses including drought, salinity, cold, heat, and infection by pathogens. In this context, when the AtPDR12 gene or homologues thereof are overexpressed (AtPDR3, AtPDR4, AtPDR10) or down-regulated (AtPDR6, AtPDR13) therein, the mutant plants are anticipated to have improved salt- or drought-resistance, thus greatly contributing to the agriculture and environment of reclaimed lands and arid regions.

1. An abscisic acid (ABA) carrier, comprising an AtPDR12 protein having an amino acid sequence of SEQ ID NO: 2 or a protein having an amino acid sequence with a homology of at least 60% with SEQ ID NO: 2.

2. The abscisic acid carrier according to claim 1, wherein the protein having an amino acid sequence with a homology of at least 60% with SEQ ID NO: 2 is selected from the group consisting of:
AtPDR3 having an amino acid sequence of SEQ ID NO: 4,
AtPDR4 having an amino acid sequence of SEQ ID NO: 6,
AtPDR6 having an amino acid sequence of SEQ ID NO: 8,
AtPDR10 having an amino acid sequence of SEQ ID NO: 10, and
AtPDR13 having an amino acid sequence of SEQ ID NO: 12.

3. A composition for the transportation of abscisic acid, comprising the abscisic acid carrier according to claim 1, or a nucleotide sequence encoding the abscisic acid carrier.

4. The composition according to claim 3, wherein the nucleotide sequence is selected from the group consisting of SEQ ID NO: 1, SEQ ID NO: 3, SEQ ID NO: 5, SEQ ID NO: 7, SEQ ID NO: 9, and SEQ ID NO: 11.

5. A composition for improving resistance of plants to salinity or drought, comprising the abscisic acid carrier according to claim 1, or a nucleotide sequence encoding the abscisic acid carrier.

6. The composition according to claim 5, wherein the nucleotide sequence is selected from the group consisting of SEQ ID NO: 1, SEQ ID NO: 3, SEQ ID NO: 5, SEQ ID NO: 7, SEQ ID NO: 9, and SEQ ID NO: 11.

7. A recombinant vector, comprising:
a promoter which can be expressed in plant cells; and
a nucleotide sequence, operably linked to the promoter, coding for an AtPDR12 protein having an amino acid sequence of SEQ ID NO: 2 or a protein having an amino acid sequence with a homology of at least 60% with SEQ ID NO: 2.

8. The recombinant vector according to claim 7, wherein the protein having an amino acid sequence with a homology of at least 60% with SEQ ID NO: 2 is selected from the group consisting of AtPDR3 having an amino acid sequence of SEQ ID NO: 4, AtPDR4 having an amino acid sequence of SEQ ID NO: 6, AtPDR6 having an amino acid sequence of SEQ ID NO: 8, AtPDR10 having an amino acid sequence of SEQ ID NO: 10, and AtPDR13 having an amino acid sequence of SEQ ID NO: 12.

9. The recombinant vector according to claim 7, wherein the nucleotide sequence is selected from the group consisting of SEQ ID NO: 1, SEQ ID NO: 3, SEQ ID NO: 5, SEQ ID NO: 7, SEQ ID NO: 9, and SEQ ID NO: 11.

10. A transgenic plant, plant cell, or part of a plant that is transformed with the recombinant vector according to any one of claims 7.

11. A method for generating a transgenic plant, comprising:
constructing the recombinant vector according to claim 7; and
introducing the recombinant vector into a plant cell or a plant tissue.

12. A method for generating a plant improved in ABA transport or resistance to salinity or drought, comprising:
constructing a recombinant vector comprising a promoter, which can be expressed in plant cells and a nucleotide sequence, operably linked to the promoter, coding for an AtPDR12 protein having an amino acid sequence of SEQ ID NO: 2 or a protein having an amino acid sequence with an homology of at least 60% with SEQ ID NO: 2.; and
introducing the recombinant vector into a plant cell or a plant tissue.

13. (canceled)

14. The method according to claim 12, wherein the protein having an amino acid sequence with a homology of at least 60% with SEQ ID NO: 2 is selected from the group consisting of AtPDR3 having an amino acid sequence of SEQ ID NO: 4, AtPDR4 having an amino acid sequence of SEQ ID NO: 6, AtPDR6 having an amino acid sequence of SEQ ID NO: 8, AtPDR10 having an amino acid sequence of SEQ ID NO: 10, and AtPDR13 having an amino acid sequence of SEQ ID NO: 12.

15. The method according to claim 12, wherein the nucleotide sequence is selected from the group consisting of SEQ ID NO: 1, SEQ ID NO: 3, SEQ ID NO: 5, SEQ ID NO: 7, SEQ ID NO: 9, and SEQ ID NO: 11.

16. The method according to claim 12, wherein when the nucleotide sequence is a coding sequence for a protein selected from the group consisting of AtPD12 of SEQ ID NO: 2, AtPDR3 of SEQ ID NO: 4, AtPDR4 of SEQ ID NO: 6, AtPDR10 of SEQ ID NO: 10, or is selected from the group consisting of SEQ ID NO: 1, SEQ ID NO: 3, SEQ ID NO: 5, and SEQ ID NO: 9, the recombinant vector contains a strong promoter or an enhancer to induce overexpression of the nucleotide sequence.

17. The method according to claim 16, wherein the strong promoter or enhancer is selected from the group consisting of a CMV35S promoter, an actin promoter, a ubiquitin promoter, a rubisco promoter, an RD29A promoter, a 35s promoter enhancer, an AtADH 5′-UTR enhancer, and an OsADH 5′-UTR enhancer.

18. The method according to claim 12, wherein when the nucleotide sequence codes for AtPDR6 of SEQ ID NO: 8 or AtPDR13 of SEQ ID NO: 12, or is selected from the group consisting of SEQ ID NO: 7 and SEQ ID NO: 11, the recombinant vector contains siRNA which binds complementary to the nucleotide sequence or an antisense RNA, T-DNA or an endogenous transposon is inserted into the nucleotide sequence, or a modification occurs on the full length or a part of the nucleotide sequence, whereby the expression of nucleotide sequence is induced to lose its function and thus to be down-regulated.

19. The transgenic plant, plant cell, or part of plant according to claim 10, wherein the plant is selected from the group consisting of onion, carrot, cucumber, olive, sweet potato, potato, Chinese cabbage, radish, lettuce, broccoli, tobacco, petunia, sunflower, Brassica napus, leaf-mustard, Arabidopsis thaliana, Brassica campestris, Brassica juncea, nicotiana tabacum, Betula platyphylla, poplar, cross-bred poplar, and Betula schmidtii.
